(19) United States
(12) Patent Application Publication
(10) Pub. No.: US 2025/0222062 A1
(43) Pub. Date: Jul. 10, 2025

(54) PEPTIDE INHIBITORS TARGETING THE TBL1-BETA-CATENIN COMPLEX

(71) Applicant: THE TRANSLATIONAL GENOMICS RESEARCH INSTITUTE, Phoenix, AZ (US)

(72) Inventors: Sunil Sharma, Phoenix, AZ (US); Soldi Raffaella, Phoenix, AZ (US)

(21) Appl. No.: 18/849,464

(22) PCT Filed: Mar. 24, 2023
(86) PCT No.: PCT/US2023/064958

§ 371 Date: Sep. 20, 2024

Related U.S. Application Data
(60) Provisional application No. 63/323,496, filed on Mar. 24, 2022.

(51) Int. Cl. A61K 38/04

(52) U.S. Cl. CPC A61K 38/04

(57) ABSTRACT

The present invention relates to a method of treating cancer in a subject, the method comprising: administering to the subject a therapeutically effective amount of a pharmaceutical composition comprising a first peptide, wherein the first peptide comprises a first amino acid sequence selected from the group consisting of SEQ ID NOs: 1-33, 52-65, and function-conservative variants thereof. Also provided is a pharmaceutical composition for the treatment of cancer, the pharmaceutical composition comprising: first peptide, wherein the first peptide comprises a first amino acid sequence selected from the group consisting of SEQ ID NOs: 1-33, 52-65, and function-conservative variants thereof.

### REFERENCE TO RELATED APPLICATIONS

This application claims priority to U.S. Provisional Application No. 63/323,496, filed Mar. 24, 2022, entitled “Peptide Inhibitors Targeting the TBL-1-BETA-Catenin Complex”; the contents of which are incorporated herein by reference thereto in its entirety.

### INCORPORATION-BY-REFERENCE OF MATERIAL ELECTRONICALLY FILED

The official copy of the sequence listing is submitted electronically in ST.26 XML format having the file name “91482-254WO-PCT_SeqList.xml” created on Mar. 23, 2023, and having a size of 59,419 bytes, and is filed concurrently with the specification. The Sequence Listing S T.26 XML file is part of the specification and is herein incorporated by reference in its entirety.

### TECHNICAL FIELD

The present invention relates generally to novel proteins and methods of peptide preparation and use as therapeutic or prophylactic agents, for example, in the treatment of cancer.

### BACKGROUND

Cancer is a group of diseases involving abnormal cell growth with a potential to spread to various parts of the body. Hundreds of types of cancers affect humans, and millions of people have been diagnosed and millions more are being diagnosed every year. The most common types of cancers include lung cancer, breast cancers, prostate cancers, colorectal cancers, among others. Treatment for cancers includes surgery, radiation therapy, chemotherapy, immunotherapy, hormone therapy, and stem cell replacement. Treatment options can be invasive and have a variety of undesirable side effects.

β-catenin, an important protein involved in the regulation and coordination of cell activity, is of particular interest in the quest to cure cancer. More than 60% of all cancers are estimated to be β-catenin-driven cancers. Expression of β-catenin is regulated through the ‘Wnt’ signaling pathway. Misregulation of this signaling pathway can lead to excess cell proliferation and tumor development. As β-catenin is the central player of the canonical Wnt signaling and it is frequently mutated in cancers, it is crucial that we develop therapeutics specifically targeting β-catenin.

Many small-molecule inhibitors of Wnt/β-catenin signaling have been identified, but none of them has been approved for the clinical use.

Accordingly, while the scientific community has made progress in this field, there remains a need in the art for improved compounds and methods for prevention and treatment of cancer.

### SUMMARY

A need exists for a therapeutic strategy for treatment of disease, including cancer. The present disclosure includes compounds (i.e., peptides) and methods for preventing and treating cancer in a subject by inhibiting formation of the complex between Transducin Beta-like protein 1 (TBL1) and beta-catenin (β-catenin).

In some aspects, the present invention provides a method of treating cancer in a subject, the method comprising: administering to the subject a therapeutically effective amount of a pharmaceutical composition comprising a first peptide, wherein the first peptide comprises a first amino acid sequence selected from the group consisting of SEQ ID NOs: 1-33, 52-65, and function-conservative variants thereof. In certain embodiments, the pharmaceutical composition comprises a first peptide, the first peptide being in an amount sufficient to disrupt the interaction between TBL1 and β-catenin.

In another aspect, the pharmaceutical composition further comprises a second peptide having a second amino acid sequence selected from the group consisting of SEQ ID NOs: 1-33, 52-65, and function-conservative variants thereof; with the proviso that the first peptide and the second peptide do not comprise the same amino acid sequence.

In another aspect, the pharmaceutical composition further comprises a third peptide having a third amino acid sequence selected from the group consisting of SEQ ID NOs: 1-33, 52-65, and function-conservative variants thereof; with the proviso that the first peptide, the second peptide, and the third peptide do not comprise the same amino acid sequence. In one aspect, the first peptide, the second peptide, and the third peptide are each less than 50 amino acids in length. In certain aspects, the function-conservative variants individually consist of a sequence that differs from any one of SEQ ID NOs: 1-33 and 52-65 by 1, 2, 3, 4, or 5 amino acids.

In some embodiments, each of the first, second, and third peptides is selected from peptides comprising one of SEQ ID NOs: 1-33 and 52-56, wherein each of the first, second and third peptides differs from the other two peptides in amino acid sequence.

In some aspects, the first peptide, the second peptide, and the third peptide individually comprise an amino acid sequence selected from the group consisting of SEQ ID NOs: 52-56.

In one aspect, the first peptide comprises an amino acid sequence of SEQ ID NO: 52.

In other aspects, the pharmaceutical composition inhibits beta-catenin (β-catenin) association with Transducin Beta-like protein 1 (TBL1) to disrupt formation of a β-catenin:TBL1 complex in the subject.

In some aspects, the cancer is a Wnt/β-catenin active cancer. In other aspects, the method further comprises determining that the subject has a Wnt/β-catenin active cancer. In certain aspects, the cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer. In another aspect, the subject is human.

In yet other aspects, administering the pharmaceutical composition to the subject is selected from the group consisting of intranasal administration, inhalational administration, intravenous administration, oral administration, and parenteral administration. In some aspects, the pharmaceutical composition comprises one or more pharmaceutically acceptable excipients.

In another aspect, the present invention relates to a method of treating a subject having a disease, the method comprising the step of administering to the subject a therapeutically effective amount of a pharmaceutical composition comprising a first peptide having at least 80% homology to a first amino acid sequence selected from the group consisting of SEQ ID NOs: 1-33 and 52-56.

In one aspect, the first peptide has at least 90% homology to the first amino acid sequence selected from the group consisting of SEQ ID NOs: 1-33 and 52-65. In another aspect, the first peptide has at least 95% homology to the first amino acid sequence selected from the group consisting of SEQ ID NOs: 1-33 and 52-65.

In yet another aspect, the pharmaceutical composition further comprises a second peptide having at least 80% homology to a second amino acid sequence selected from the group consisting of SEQ ID NOs: 1-33 and 52-65, or at least 90% homology to the second amino acid sequence selected from the group consisting of SEQ ID NOs: 1-33 and 52-65, or at least 95% homology to the second amino acid sequence selected from the group consisting of SEQ ID NOs: 1-33 and 52-65; with the proviso that the first peptide and the second peptide do not comprise the same amino acid sequence.

In another aspect, the pharmaceutical composition further comprises a third peptide having at least 80% homology to a third amino acid sequence selected from the group consisting of SEQ ID NOs: 1-33 and 52-65, or at least 90% homology to the third amino acid sequence selected from the group consisting of SEQ ID NOs: 1-33 and 52-65, or at least 95% homology to the third amino acid sequence selected from the group consisting of SEQ ID NOs: 1-33 and 52-65; with the proviso that the first peptide, the second peptide, and the third peptide do not comprise the same amino acid sequence.

In some aspects, the first peptide, the second peptide, and the third peptide individually comprise an amino acid sequence having at least 80% homology to sequence selected from the group consisting of SEQ ID NOs: 52-56. In one aspect, the first peptide comprises an amino acid sequence having at least 80% homology to SEQ ID NO: 52. In some aspects, the pharmaceutical composition comprises a peptide with at least 80%, 81%, 82%, 83%, 84%, 85%, 86%, 87%, 88%, 89%, 90%, 91%,92%, 93%, 94%, 95%, 96%, 97%, 98%, 99%,or 100% homology to an amino acid sequence selected from the group consisting of SEQ ID NOs: 1-33 and 52-56.

In some aspects, the disease is a Wnt/β-catenin active cancer. In other aspects, the method further comprises determining that the subject has a Wnt/β-catenin active cancer.

In yet other aspects, the present invention provides a pharmaceutical composition for the treatment of cancer, the pharmaceutical composition comprising: a first peptide, wherein the first peptide comprises a first amino acid sequence selected from the group consisting of SEQ ID NOs: 1-33, 52-65, and function-conservative variants thereof. In other aspects, the pharmaceutical composition further comprises a second peptide, wherein the second peptide comprises a second amino acid sequence selected from the group consisting of SEQ ID NOs: 1-33, 52-65, and function-conservative variants thereof; with the proviso that the first peptide and the second peptide do not comprise the same amino acid sequence.

In other aspects, the pharmaceutical composition further comprises a third peptide, wherein the third peptide comprises a third amino acid sequence selected from the group consisting of SEQ ID NOs: 1-33, 52-65, and function-conservative variants thereof; with the proviso that the first peptide, the second peptide, and the third peptide do not comprise the same amino acid sequence. In one aspect, the pharmaceutical composition further comprises at least one pharmaceutically acceptable carrier.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIG. 1A depicts the canonical β-catenin dependent WNT pathway. FIG. 1B depicts a peptide approach as a therapeutic targeting the TBL1/β-catenin complex.

FIG. 2 depicts the Armadillo domain of β-catenin protein (SEQ ID NO: 37).

FIG. 3 depicts the Sigma Peptide Library design and calculator tool with an entire protein sequence (SEQ ID NO: 38) and peptide sequence output (SEQ ID NOs: 39-51).

FIG. 4 depicts the Sandwich ELISA workflow.

FIGS. 5A and 5B depict the results and optimization of a sandwich ELISA assay.

FIG. 6 depicts an example of the results of the peptide library screening with single peptides and combinations of two or three peptides.

FIG. 7 depicts an example of the results of the peptide library screening with single peptides demonstrating the efficacy of peptides 4, 5, 6, 17, 18, and 19.

FIGS. 8A and 8B depict a sandwich ELISA for peptides screening in HCT15 and SW480 colon cancer cell lysates with arrows indicating peptide combinations selected for further evaluation.

FIGS. 9A, 9B, 9C, 9D and 9E depict a sandwich ELISA for peptides screening colon cancer cell lysates in which combinations of peptides were assayed at varying concentrations to demonstrate dose-dependent inhibition of the formation of the TBL1/β-catenin complex.

FIG. 10A to 1° F. depict five longer peptide sequences (Peptides 1-5 which are SEQ ID NOs: 52-56 respectively) and their properties. These longer peptides were evaluated for binding with TBL1 based on the efficacy of combinations of peptides.

FIG. 11 depicts a homologous competitive ELISA assay performed with increasing logarithmic concentrations of peptides (i.e., Peptides 1, 4, and 5). The peptides selected are able to displace β-catenin from TBL1 complex in a dose dependent manner.

FIG. 12 depicts an in silico analysis of a hit peptide binding on TBL1 pockets performed with PIPER-pepdock software.

FIG. 13 depicts a pull-down assay with TBL1 in the SW480 colorectal cancer (CRC) cell line transfected with the selected peptides.

FIG. 14 depicts a Western blot for TBL1 and associated β-catenin after the pull-down assay with TBL1 in the SW480 colorectal cancer (CRC) cell line transfected with five selected peptides (i.e., Peptides 1-5) to show disruption of the complex by the selected peptides.

FIGS. 15A and 15B depict the results of a TOPFlash TCF/LEF reporter assay monitoring Wnt/β-catenin signaling pathway activity in cells expressing BC2059 (i.e., positive control) and Peptides 1-5.

FIG. 16 depicts the results of an ELISA assay using DANSYL tagged peptides (i.e., Peptides 1-5) to measure association with TBL1.

FIG. 17 presents a TOPFlash assay to determine transcription activity downstream TCF/LEF: HEK293 cells containing a luciferase downstream TCF/LEF were LiCl was administered 16 hours prior to treatment to stimulate the Wnt pathway. Then the cells were treated with recombinant Wnt3a to stimulate transcription and peptide at 5 μM or BC-2059 at 0.5 μM for 6 hours before luciferase expression was quantified with the One-Step Luciferase assay system. We identified that peptides 2, 3, and 5 all have decreased levels of luciferase after treatment identifying that these peptides disrupt β-catenin's activity as a transcriptional coactivator.

FIG. 18 presents wash out TOPFlash at 8, 12,18, and 24 hour time points: to investigate the effects of peptide 3 over time with a washout TOPFlash assay. To do this we followed the same procedure previously stated but performed washouts at 8, 12, 18, and 24 hour time points. This experiment was completed in duplicate. We identified that if the peptide is washed out after 8 and 12 hours, we see an increase in luciferase produced compared to the non washout control. This identifies that peptide 3 is specific and effective at blocking transcription downstream TCF/LEF.

FIG. 19 shows immunofluorescence images taken to identify if CPP peptides penetrate SW480 cells. SW480 cells were seeded onto glass slides then treated with 5 μM peptide 4 and incubated for 6 hours. After incubation the cells were washed and fixed then treated with Flag antibody overnight. After this a green fluorescent a secondary antibody was incubated. The cells were washed and mounted with DAPI mounting media then visualized on a confocal microscope. CPP-peptides do penetrate the cells, however they appear to form aggregates which we hypothesize is due to instability of peptide structure.

FIG. 20 illustrates that to identify the localization of the peptides into SW480 cells, we repeated the immunofluorescence as previously described but added and additional actin stain to aid in identification of peptide localization. These peptides were also incubated for 24 hours before fixation to allow more time to penetrate. We found that the peptides are found within the cells however they appear to cluster in the cytoplasmic extensions.

FIG. 21 presents certain peptide specifications. Due to the concerns of our peptide stabilization, we have optimized the peptides to be shorter and to have a cyclized CPP. We believe that these modifications will aid in the peptide stability and will make our peptides more effective.

FIG. 22 presents TOPFlash assay to determine transcription activity downstream TCF/LEF: HEK293 cells containing a luciferase downstream TCF/LEF were with LiCl 16 hours prior to treatment to stimulate the Wnt pathway. Then they were treated with recombinant Wnt3a to stimulate transcription and peptide 16 (short) and 2 (long) at 5 μM or BC-2059 at 0.5 μM for 6 hours before luciferase expression was quantified with the One-Step Luciferase assay system. Peptides 16 showed higher efficacy to promote decreased levels of luciferase after treatment compared to peptide 2, though the difference is not statistically significant.

FIG. 23 presents TOPFlash assay results to determine transcription activity downstream TCF/LEF dependency to peptides: HEK293 cells containing a luciferase downstream TCF/LEF were with LiCl 16 hours prior to treatment to stimulate the Wnt pathway. Then they were treated with recombinant Wnt3a to stimulate transcription and peptide 16 (short) and 2 (long) at different concentrations (300 μM-0.01 μM) for 6 hours before luciferase expression was quantified with the One-Step Luciferase assay system. TCF/LEF transcriptional activity decreases with treatment with increased concentration of peptides 2 and 16.

FIG. 24 shows the cytotoxic effect of peptides 2 and 6 on different cell lines (H358 (lung cancer) HCT15, HCT116, and SW480 (colon cancer) characterized by WNT pathway activation/dependency. Cells were incubated with increasing concentrations of peptide 16 (short) and 2 (long) at different concentrations (300 μM-0.01 μM) for 72 hours. Cell viability was assessed by CellTiter Glo.

FIG. 25 illustrates that peptide 16 promotes polyubiquitination of β-catenin. HCT 15 cells were treated with 20 mM LiCl, the universal activator of Wnt signaling, in presence/absence of 0.5 M BC-2059 or 5 μM peptide 16 for 6 hours. Harvested cells were subjected to lysis and samples were processed for enrichment of polyubiquitinated protein conjugates with the Ubiquitin Enrichment Kit (Thermo Scientific) according to the manufacturer's protocol. Samples were resolved in 4-12% SDS-PAGE (Invitrogen). Western blot analysis was performed using β-catenin antibody (BD Biosciences) and Odyssey Imager (Li-Cor Biosciences).The results show that peptide 16 promotes polyubiquitination of (3-catenin.

### DETAILED DESCRIPTION

The Wnt signaling pathway and its downstream transcriptional activator β-catenin are involved in critical cellular processes including oncogenesis. Peptides are superior as therapeutics to small molecules in interacting with β-catenin. To achieve efficient elimination of β-catenin, we designed novel peptides that interfere with β-catenin:TBL1 complex formation, which is crucial for the activation of Wnt signaling β-catenin dependent. This disclosure is the first to block Wnt signaling via peptides against β-catenin: TBL1 complex, highlighting the potential of the peptides as anew class of promising agents against the diseases caused by overactivation of Wnt/β-catenin signaling.

In the absence of Wnt-signaling, β-catenin is recognized by the ubiquitin/proteasome machinery and degraded in the cytoplasm (1). In contrast, active Wnt-signaling leads to β-catenin association with Transducin Beta-like protein 1 (TBL1) and Transducin Beta-like related protein 1 (TBLR1), with subsequent translocation to the nucleus where it drives transcription of Wnt-regulated genes (2). Recent studies showed interaction of β-catenin with TBL1 protein family is critical for protecting β-catenin from ubiquitin-mediated degradation (3) and Wnt/β-catenin-mediated transcription (4). Depletion of TBL1/TBLR1 significantly inhibits Wnt/β-catenin-induced gene expression and blocks growth of tumor cells in vitro and in vivo (4). Moreover, TBL1, interacts with TCF7L2, an interaction enhanced upon Wnt activation (4), suggesting that the formation of TBL1/β-catenin complex is a key regulatory point downstream of Wnt pathway activation (2).

The development of inhibitors targeting the TBL1-β-catenin complex may improve the conventional treatment of several cancers that harbor activating mutations in the Wnt pathway.

To this purpose, we designed β-catenin peptides library and optimization of the screening assay. In cancer, active Wnt-signaling leads to β-catenin association with Transducin Beta-like protein 1 (TBL1) and Transducin Beta-like related protein 1 (TBLR1). This association is crucial for β-catenin translocation to the nucleus where it drives transcription of Wnt-regulated genes, and prevents β-catenin from nuclear ubiquitin mediated degradation. Modulation of TBL1/β-catenin complex is a key regulatory point downstream of Wnt pathway activation. We hypothesized that incubation with peptides that interfere with the binding site between TBL1 and β-catenin may lead to disruption of the complex, resulting in inhibition of the WNT-dependent signaling. To this purpose, we designed a peptide library that covers the region on β-catenin protein that we hypothesized interacts with TBL1. The area of interest is a core region of beta-catenin, composed of 12 copies of a 42 amino acid sequence motif known as an armadillo repeat. In particular, TBL1 is known to interact with β-catenin in the region included between residues 143-500. By using the Sigma Peptide Library Design and Calculator Tool, we designed 33 overlapping peptides (Length of each peptide=20 amino acids; Each peptide overlap 8 amino acids with the previous one) to ensure complete coverage of the area of interest.

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

The terms “peptide,” “polypeptide,” and “protein” are used interchangeably herein to refer to polymers of amino acids of any length, chemically or biochemically modified or derivatized amino acids, and polypeptides having modified peptide backbones. These terms also include proteins that are post-translationally modified through reactions that include glycosylation, acetylation and phosphorylation. The term “at least a portion” of a polypeptide means a portion having the minimal size characteristics of such sequences, or any larger fragment of the full-length molecule, up to and including the full-length molecule. For example, a portion of a polypeptide may be 4 to 15 amino acids, or may be 4 amino acids, 5 amino acids, 6 amino acids, 7 amino acids, and so on, up to a full-length polypeptide. A portion of a polypeptide useful as an epitope may be as short as 4 amino acids. A portion of a polypeptide that performs the function of the full-length polypeptide would generally be longer than 4 amino acids.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified. Unnatural amino acids are not encoded by the genetic code and can, but do not necessarily have the same basic structure as a naturally occurring amino acid. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to by either the three letter symbols or by the one-letter symbols recommended by the IUPAC, the IUAPC letter code are as follows: G=Glycine; A=Alanine; L=Leucine; M=Methionine; F=Phenylalanine; W=Tryptophan; K=Lysine; Q=Glutamine; E=Glutamic Acid; S=Serine; P=Proline; V=Valine; I=Isoleucine; C=Cysteine; Y=Tyrosine; H=Histidine; R=Arginine; N=Asparagine; D=Aspartic Acid; T=Threonine.

The terms “homologous” and “similar” refer to the relationship between proteins that possess a “common evolutionary origin,” including proteins from superfamilies (e.g., the immunoglobulin superfamily) and homologous proteins from different species. Such proteins (and their encoding genes) have sequence homology, as reflected by their sequence similarity, whether in terms of percent similarity or the presence of specific residues or motifs as conserved positions. In a specific embodiment, two peptide sequences are “substantially homologous or similar” when at least about 80%, or at least about 90%, or at least about 95) of the amino acids match over the defined lengths of the amino acid sequences.

The term “variants” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Variants may include individual substitutions, deletions or additions to a nucleic acid, peptide, polypeptide, or protein sequence which alters, adds or deletes a single amino acid or a small percentage of amino acids in the encoded sequence.

“Function-conservative variants” or “functionally equivalent variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein so that the percent protein or amino acid sequence similarity between any two proteins of similar function may vary and may be, for example, from 70% to 99% as determined according to an alignment scheme. A “variant” also includes a polypeptide which has at least 60% amino acid identity as determined by BLAST or FASTA algorithms, preferably at least 75% most preferably at least 85%, and even more preferably at least 90%, and still more preferably at least 95%, and which has the same or substantially similar properties or functions as the native or parent protein to which it is compared. A particular variant is a “gain-of-function” variant, meaning a polypeptide variant in which the change of at least one given amino acid residue in a protein or enzyme improves a specific function of the polypeptide, including, but not limited to protein activity. The change in amino acid residue can be replacement of an amino acid with one having similar properties.

The term “functionally equivalent” thus includes any equivalent of a specific peptide obtained by altering the amino acid sequence, for example by one or more amino acid deletions, substitutions or additions. Amino acid substitutions may be made, for example, by point mutation of the DNA encoding the amino acid sequence.

In some aspects, the functional equivalent is at least 80% homologous to the corresponding protein. In a particular embodiment, the functional equivalent is at least 90% homologous as assessed by any conventional analysis algorithm such as for example, the Pileup sequence analysis software (Program Manual for the Wisconsin Package, 1996).

“Small molecule,” as used herein, means a molecule less than 5 kilodaltons, more typically, less than 1 kilodalton.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex.

As used herein, the term “specific binding” refers to the specificity of a binder, e.g., a protein or an antibody, such that it preferentially binds to a target, such as a polypeptide antigen, a receptor, or an antibody. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments, peptides, or fusion peptides that are specific for or bind specifically to a target bind to the target with higher affinity than binding to other non-target substances. Also preferably, binders, antibodies or antibody fragments, peptides, or fusion peptides that are specific for or bind specifically to a target avoid binding to a significant percentage of non-target substances, e.g., non-target substances present in a testing sample. The binding affinity of an antibody to a target antigen, antigenic fragment, peptide, or fusion peptide, comprising the cognate epitope can be readily determined using any of a number of methods available in the art including, but not limited to, enzyme linked immunosorbent assay (ELISA). In some embodiments, binders, antibodies or antibody fragments, peptides, or fusion peptides of the present disclosure avoid binding greater than about 90% of non-target substances, although higher percentages are clearly contemplated and preferred. For example, binders, antibodies or antibody fragments, or peptides, of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments, or peptides of the present disclosure avoid binding greater than about 10%, 20%, 30%, 40%, 50%, 60%, or 70%, or greater than about 75%, or greater than about 80%, or greater than about 85% of non-target substances.

A target or a marker may be any molecular structure produced by a cell, expressed inside the cell, accessible on the cell surface, or secreted by the cell. A marker may be any protein, carbohydrate, fat, nucleic acid, catalytic site, or any target of these such as an enzyme, glycoprotein, cell membrane, virus, cell, organ, organelle, or any uni- or multimolecular structure or any other such structure now known or yet to be disclosed whether alone or in combination. A target may also be called a marker and the terms are used interchangeably.

A target may be represented by the sequence of amino acids, or sequence of one or more strands of a nucleic acid from which it may be derived. For example, a target may be represented by a protein sequence. Alternatively, a target may be represented by a nucleic acid sequence, the protein or peptide or the fragments thereof encoded by the nucleic acid sequence.

Examples of such nucleic acids include both single stranded and double stranded nucleic acid sequences including miRNA, tRNA, siRNA, mRNA, cDNA, or genomic DNA sequences including complimentary sequences. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The term “target” further encompasses the products (i.e., proteins) of the gene or a gene allele thereof, whose expression or activity is directly or indirectly associated with a particular phenotype or cellular condition, or physiological characteristic.

In some embodiments, the pharmaceutical composition includes one or more peptides or fragments, as described herein, together with a pharmaceutically acceptable carrier, diluent or excipient. In the preparation of the pharmaceutical compositions comprising the peptides described in the teachings herein, a variety of vehicles, vectors, excipients and routes of administration may be used. The pharmaceutical compositions will generally comprise a pharmaceutically acceptable carrier and a pharmacologically (or therapeutically) effective amount of the peptides.

The pharmaceutical compositions described herein may be administered by any means that enables the active agent to reach the agent's site of action in the body of the subject. The dosage administered varies depending upon factors, such as: pharmacodynamic characteristics; mode and route of administration; age, health, and weight of the recipient subject; nature and extent of symptoms; concurrent treatments; and frequency of treatment.

As used herein, the terms “administration” and “administering” of an agent to a subject include any route of introducing or delivering the agent to a subject to perform its intended function. Administration can be carried out by any suitable route, including intravenously, intramuscularly, intraperitoneally, inhalationally, intranasally, or subcutaneously. Administration includes self-administration and the administration by another.

The term “effective amount” or “therapeutically effective amount” refers to that amount of an agent or combination of agents as described herein that is sufficient to affect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “patient” or “subject” refers to any organism to which a provided composition is or may be administered, e.g., for experimental, diagnostic, prophylactic, cosmetic, and/or therapeutic purposes. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human.

In some embodiments, a patient is suffering from or susceptible to one or more disorders or conditions. In some embodiments, a patient displays one or more symptoms of a disease, disorder or condition. In some embodiments, a patient has been diagnosed with one or more diseases, disorders or conditions. In some embodiments, the disorder or condition is or includes cancer, or the presence of one or more tumors.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

An effective amount of a therapeutic peptide is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. In some aspects, a therapeutic regimen is carried out by identifying a mammal, e.g., a human patient suffering from a cancer that has a Wnt/β-catenin active cancer using standard methods. For example, the subject has a Wnt/β-catenin activating mutation.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic peptides described herein are formulated into compositions for other routes of administration utilizing conventional methods. For example, the therapeutic peptides are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The peptide is administered in the form of a hard-shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Design of a Peptide Library for Therapeutics Targeting β-catenin/TBL1 Complex

The Wnt signaling pathway and its downstream transcriptional activator β-catenin are involved in critical cellular processes including oncogenesis. In the absence of Wnt-signaling, β-catenin is recognized by the ubiquitin/proteasome machinery and degraded in the cytoplasm (1). In contrast, active Wnt-signaling leads to β-catenin association with Transducin Beta-like protein 1 (TBL1) and Transducin Beta-like related protein 1 (TBLR1), with subsequent translocation to the nucleus where it drives transcription of Wnt-regulated genes (2). Recent studies showed interaction of β-catenin with the TBL1 protein family is critical for protecting β-catenin from ubiquitin-mediated degradation (3) and Wnt/β-catenin-mediated transcription (4). Depletion of TBL1/TBLR1 significantly inhibits Wnt/β-catenin-induced gene expression and blocks growth of tumor cells in vitro and in vivo (4). Moreover, TBL1, interacts with TCF7L2, an interaction enhanced upon Wnt activation (4), suggesting that the formation of TBL1/β-catenin complex is a key regulatory point downstream of Wnt pathway activation (2).

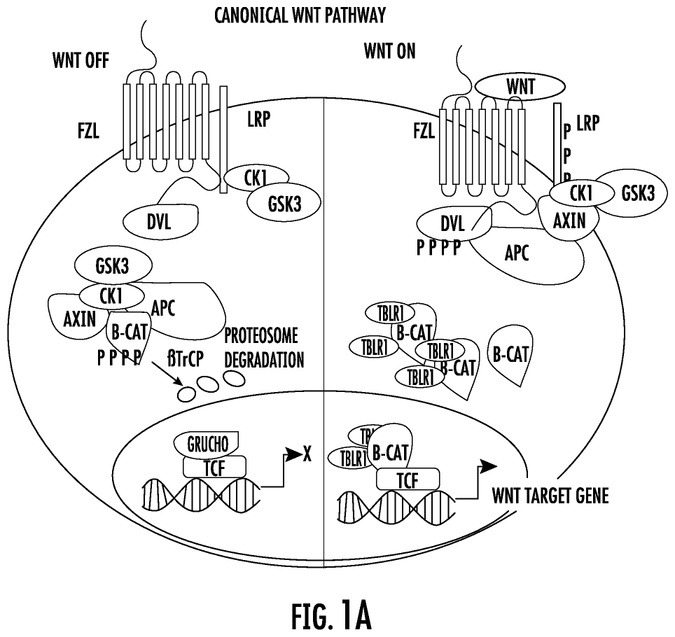

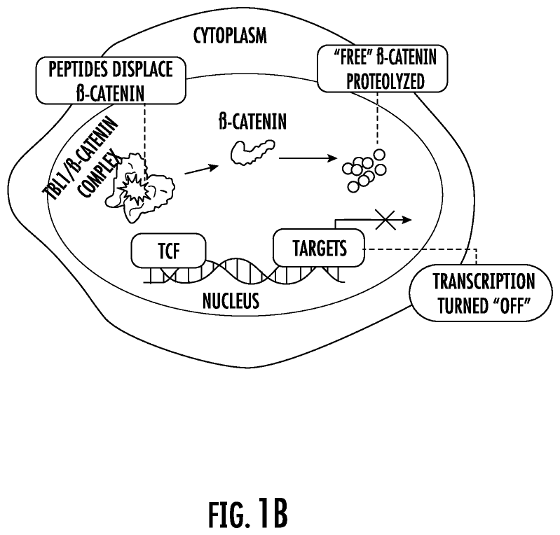

β-catenin, an important protein involved in the regulation and coordination of cell activity, is of particular interest in the quest to cure cancer. More than 60% of all cancers are estimated to be β-catenin-driven cancers. Expression of β-catenin is regulated through the ‘Wnt’ signaling pathway. Misregulation of this signaling pathway can lead to excess cell proliferation and tumor development. As illustrated by FIG. 1A, D-catenin is the central player of the canonical Wnt signaling and it is frequently mutated in cancers. Here we develop therapeutics specifically targeting β-catenin.

Many small-molecule inhibitors of Wnt/β-catenin signaling have been identified, but none of them has been approved for clinical use. Peptides are superior as therapeutics to small molecules in interacting with β-catenin. To achieve efficient elimination of β-catenin, we designed novel peptides that interfere with j-catenin:TBL1 complex formation, crucial for the activation of Wnt signaling β-catenin dependent (see FIG. 1B). The study disclosed herein is the first attempt to block Wnt signaling via peptides against the β-catenin:TBL1 complex, highlighting the potential of the peptides as a new class of promising agents against the diseases caused by overactivation of Wnt/β-catenin signaling.

The development of inhibitors targeting the TBL1-β-catenin complex may improve the conventional treatment of several cancers that harbor activating mutations in the Wnt pathway. To this purpose we designed a β-catenin peptide library and optimized a screening assay. In cancer, active Wnt-signaling leads to β-catenin association with TBL1 and TBLR1. This association is important for β-catenin translocation to the nucleus where it drives transcription of Wnt-regulated genes and prevents β-catenin from nuclear ubiquitin-mediated degradation. Modulation of TBL1/β-catenin complex is a key regulatory point downstream of the Wnt pathway activation. It was hypothesized that incubation with peptides that interfere with the binding site between TBL1 and β-catenin may lead to disruption of the complex, resulting in inhibition of the WNT-dependent signaling. To this purpose, a peptide library (TABLE 1) was designed that covers the region (TABLE 2) on β-catenin protein that we hypothesized interacts with TBL1. The area of interest is a core region of beta-catenin, composed of 12 copies of a 42 amino acid sequence motif known as an armadillo repeat (TABLE 2).

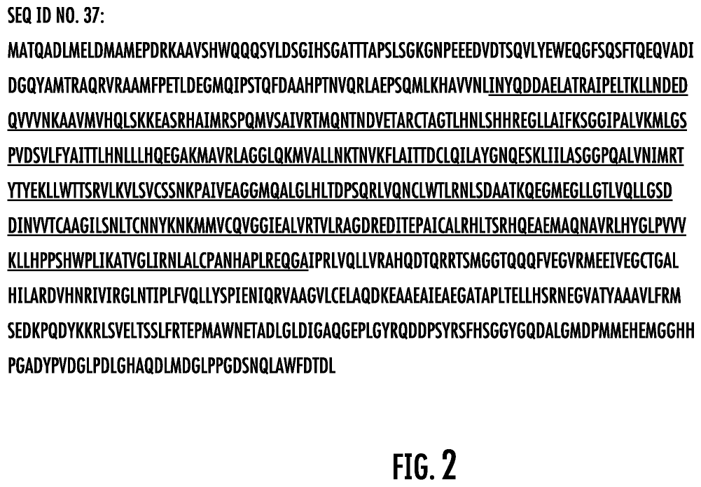

FIG. 2 shows the β-catenin Armadillo (ARM) Domain. The ARM domain of β-catenin has a typical curved structure with a positively charged groove, which creates a large binding surface for proteins. This region of β-catenin is composed of 12 α-helices having a 42 amino acid sequence motif involved in protein-protein interactions. Underlined in FIG. 2 and in SEQ ID NO: 37 is the region of interest (SEQ ID NO: 38) used to generate the peptides library in TABLE 1.

TABLE 1 shows exemplary amino acid sequences, i.e. the peptide library, for the therapeutic peptides. Several target-binding amino acid sequences are disclosed for a peptide designed for the treatment of cancer, including amino acid sequences having any one of SEQ ID NOS: 1-33. Based on the 03-catenin armadillo domain sequence, 33 peptides were designed that cover the region of the protein included between residues 143 and 500. Each of the peptides having one of SEQ ID NOS: 1-33 is 20 amino acids long and overlaps the subsequent peptide by 8 amino acids. Thus, the 33 peptides (i.e., SEQ ID NOS: 1-33) cover the armadillo domain and were designed to interact with TBL1 and break the TBL1/β-catenin complex. Peptides having SEQ ID NOS: 34-36 correspond to a portion of β-catenin outside the armadillo domain and are used as negative controls.

TABLE 1
Peptide Sequences

| Sequence | | SEQ |
|---|---|---|
| Reference | | ID |
| Name | Amino Acid Sequence | NO |
| Pep1 | INYQDDAELATRAIPELTKL | 1 |
| Pep2 | AIPELTKLLNDEDQVVVNKA | 2 |
| Pep3 | DQVVVNKAAVMVHQLSKKEA | 3 |
| Pep4 | HQLSKKEASRHAIMRSPQMV | 4 |
| Pep5 | IMRSPQMVSAIVRTMQNTND | 5 |
| Pep6 | RTMQNTNDVETARCTAGTLH | 6 |
| Pep7 | RCTAGTLHNLSHHREGLLAI | 7 |
| Pep8 | HREGLLAIFKSGGIPALVKM | 8 |
| Pep9 | GIPALVKMLGSPVDSVLFYA | 9 |
| Pep10 | VDSVLFYAITTLHNLLLHQE | 10 |
| Pep11 | HNLLLHQEGAKMAVRLAGGL | 11 |
| Pep12 | AVRLAGGLQKMVALLNKTNV | 12 |
| Pep13 | ALLNKTNVKFLAITTDCLQI | 13 |
| Pep14 | ITTDCLQILAYGNQESKLII | 14 |
| Pep15 | NQESKLIILASGGPQALVNI | 15 |
| Pep16 | GPQALVNIMRTYTYEKLLWT | 16 |
| Pep17 | TYEKLLWTTSRVLKVLSVCS | 17 |
| Pep18 | LKVLSVCSSNKPAIVEAGGM | 18 |
| Pep19 | AIVEAGGMQALGLHLTDPSQ | 19 |
| Pep20 | LHLTDPSQRLVQNCLWTLRN | 20 |
| Pep21 | NCLWTLRNLSDAATKQEGME | 21 |
| Pep22 | ATKQEGMEGLLGTLVQLLGS | 22 |
| Pep23 | TLVQLLGSDDINVVTCAAGI | 23 |
| Pep24 | VVTCAAGILSNLTCNNYKNK | 24 |
| Pep25 | TCNNYKNKMMVCQVGGIEAL | 25 |
| Pep26 | QVGGIEALVRTVLRAGDRED | 26 |
| Pep27 | LRAGDREDITEPA | 27 |
| Pep28 | LRAGDREDITEPAICALRHL | 28 |
| Pep29 | AICALRHLTSRHQEAEMAQN | 29 |
| Pep30 | QEAEMAQNAVRLHYGLPVVV | 30 |
| Pep31 | HYGLPVVVKLLHPPSHWPLI | 31 |
| Pep32 | PPSHWPLIKATVGLIRNLALC | 32 |
| Pep33 | LIRNLALCPANHAPLREQGA | 33 |
| Negative Control 1 | DVDTSQVLYEWEQGFSQSFT | 34 |
| Negative Control 2 | QGFSQSFTQEQVADIDGQYA | 35 |
| Negative Control 3 | ADIDGQYAMTRAQRVRAAMFPET | 36 |

TABLE 2

| | | SEQ |
|---|---|---|
| Sequence | | ID |
| Name | Amino Acid Sequence | NO |
| β-catenin | MATQADLMELDMAMEPDRKAAVSHWQQQSY | 37 |
| Armadillo | LDSGIHSGATTTAPSLSGKGNPEEEDVDTS | |
| Domain | QVLYEWEQGFSQSFTQEQVADIDGQYAMTR | |
| | AQRVRAAMFPETLDEGMQIPSTQFDAAHPT | |
| | NVQRLAEPSQMLKHAVVNLINYQDDAELAT | |
| | RAIPELTKLLNDEDQVVVNKAAVMVHQLSK | |
| | KEASRHAIMRSPQMVSAIVRTMQNINDVET | |
| | ARCTAGTLHNLSHHREGLLAIFKSGGIPAL | |
| | VKMLGSPVDSVLFYAITTLHNLLLHQEGAK | |
| | MAVRLAGGLQKMVALLNKTNVKFLAITTDC | |
| | LQILAYGNQESKLIILASGGPQALVNIMRT | |
| | YTYEKLLWTTSRVLKVLSVCSSNKPAIVEA | |
| | GGMQALGLHLTDPSQRLVQNCLWTLRNLSD | |
| | AATKQEGMEGLLGTLVQLLGSDDINVVTCA | |
| | AGILSNLTCNNYKNKMMVCQVGGIEALVRT | |
| | VLRAGDREDITEPAICALRHLTSRHQEAEM | |
| | AQNAVRLHYGLPVVVKLLHPPSHWPLIKAT | |
| | VGLIRNLALCPANHAPLREQGAIPRLVQLL | |
| | VRAHQDTQRRTSMGGTQQQFVEGVRMEEIV | |
| | EGCTGALHILARDVHNRIVIRGLNTIPLFV | |
| | QLLYSPIENIQRVAAGVLCELAQDKEAAEA | |
| | IEAEGATAPLTELLHSRNEGVATYAAAVLF | |
| | RMSEDKPQDYKKRLSVELTSSLFRTEPMAW | |
| | NETADLGLDIGAQGEPLGYRQDDPSYRSFH | |
| | SGGYGQDALGMDPMMEHEMGGHHPGADYPV | |
| | DGLPDLGHAQDLMDGLPPGDSNQLAWFDTD | |
| | L | |
| ARM | INYQDDAELATRAIPELTKLLNDEDQVVVN | 38 |
| Domain | KAAVMVHQLSKKEASRHAIMRSPQMVSAIV | |
| Region | RTMQNTNDVETARCTAGTLHNLSHHREGLL | |
| of | AIFKSGGIPALVKMLGSPVDSVLFYAITTL | |
| Interest | HNLLLHQEGAKMAVRLAGGLQKMVALLNKT | |
| | NVKFLAITTDCLQILAYGNQESKLIILASG | |
| | GPQALVNIMRTYTYEKLLWTTSRVLKVLSV | |
| | CSSNKPAIVEAGGMQALGLHLTDPSQRLVQ | |
| | NCLWTLRNLSDAATKQEGMEGLLGTLVQLL | |
| | GSDDINVVTCAAGILSNLTCNNYKNKMMVC | |
| | QVGGIEALVRTVLRAGDREDITEPAICALR | |
| | HLTSRHQEAEMAQNAVRLHYGLPVVVKLLH | |
| | PPSHWPLIKATVGLIRNLALCPANHAPLRE | |
| | QGA | |

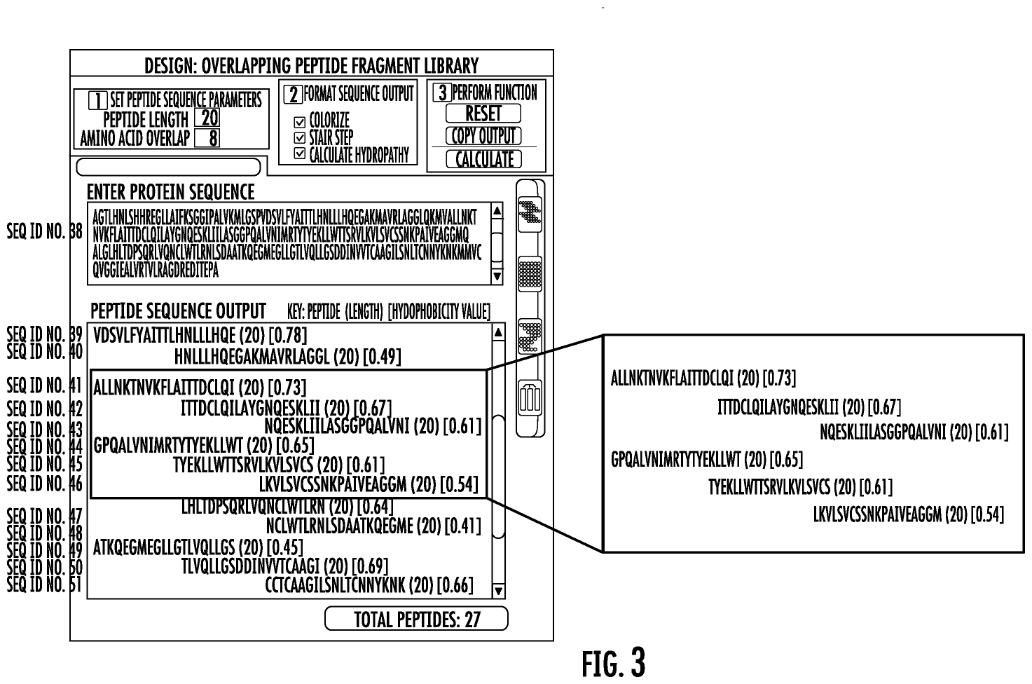

In particular, TBL1 is known to interact with β-catenin in the region included between residues 143-500. By using the Sigma Peptide Library Design and Calculator Tool (FIG. 3), we designed 33 overlapping peptides (Length of each peptide=20 amino acids; Each peptide overlap 8 amino acids with the previous one) to ensure complete coverage of the area of interest. FIG. 3 depicts the Sigma Peptide Library design and calculator tool with an entire protein sequence (SEQ ID NO: 38) and peptide sequence output (SEQ ID NOs: 39-51).

SEQ ID NO. 39:
VDSVLFAITTLHNLLLHQE
SEQ ID NO. 40:
HNLLLHQEGAKMAVRLAGGL
SEQ ID NO. 41:
ALLNKTNVKFLAITTDCLQI
SEQ ID NO. 42:
ITTDCLQILAYGNQESKLII
SEQ ID NO. 43:
NQESKLIILASGGPQALVNI
SEQ ID NO. 44:
GPQALVNIMRTYTYEKLLWT
SEQ ID NO. 45:
TYEKLLWTTSRVLKVLSVCS
SEQ ID NO. 46:
LKVLSVCSSNKPAIVEAGGM
SEQ ID NO. 47:
LHLTDPSQRLVQNCLWTLRN
SEQ ID NO. 48:
NCLWTLRNLSDAATKQEGME
SEQ ID NO. 49:
ATKQEGMEGLLGTLVOLLGS
SEQ ID NO. 50:
TLVQLLGSDDINVVTCAAGI
SEQ ID NO. 51:
VVTCAAGILSNLTCNNYKNK

### Example 2. Initial Screening of Peptide Library for In Vitro Activity

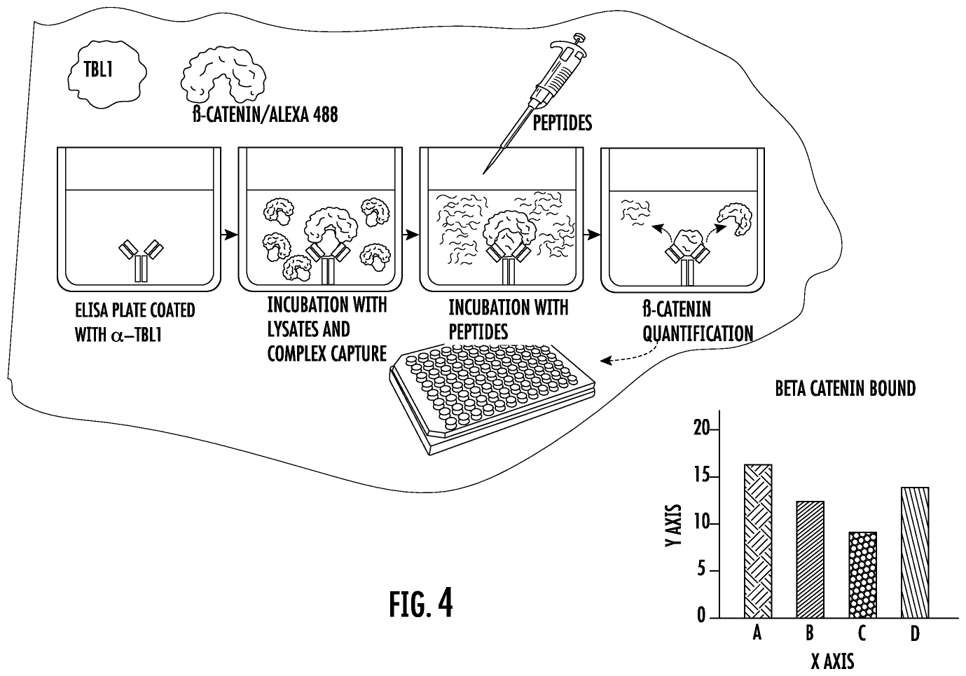

To identify the peptide or peptides able to disrupt the interaction between TBL1 and (3-catenin, the sandwich ELISA approach was used (see FIG. 4). In this assay TBL1/β-catenin complex from lysates of a colon cancer cell with active Wnt signaling was captured in a Protein G ELISA 96-well plate coated with antibody that recognizes TBL1. To ensure activation of Wnt signaling, the cells were pre-treated with 20 mM LiCl for 12 h a 37° C. The bound complex was then exposed to peptides from the library either as single peptides or in a combination of 2 or 3 peptides to ensure coverage of the potential binding site between TBL1 and β-catenin. After washing away unbound proteins, the plates were incubated with β-catenin antibodies labeled with ALEXA FLUOR® 488 dye, and the level of β-catenin associated to TBL1 was quantified by a plate reader that detects the ALEXA FLUOR® 488 fluorescence probe. As a form of TBL1/β-catenin complex, we used colon cancer cell lines HCT15 and SW480. All the experiments were conducted in triplicate to ensure statistical significance. To reduce variability the assays were performed by using the EPMOTION® liquid handler. We designed a program that controls the peptide seeding into the ELISA plate wells: 1, 2 or 3 peptides per well, with overlap of one peptide in each well.

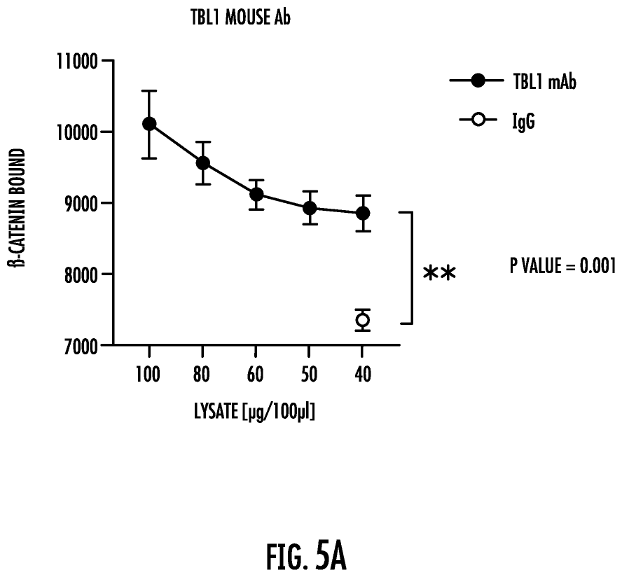

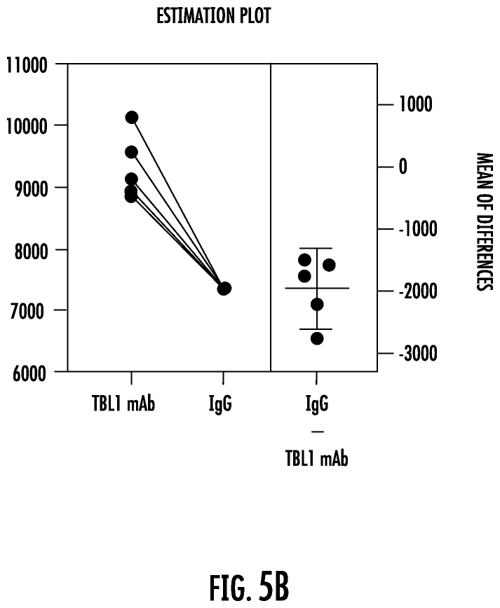

FIGS. 5A and 5B show the results and optimization of the sandwich ELISA assay and a standard curve. A TBL1 mouse/β-catenin rabbit/α-Rb-488 combination showed a gradual decrease in the ALEXA FLUOR® 488 emission as SW480 lysates decrease, and the control well (hIgG) shows a dramatic decrease in emission with the same amount of protein as the lowest well.

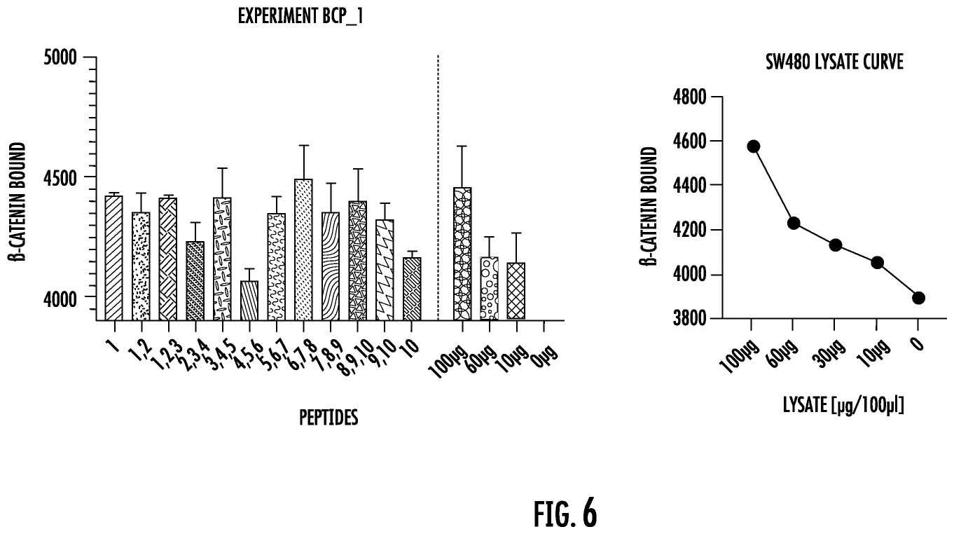

FIG. 6 shows an example of results of the peptides library screening. All wells in the screening were loaded with 1 mg/ml of lysate and incubated at 37° C. for 1 hour. After a wash, peptide combinations were added at a concentration of 10 NM for each peptide and incubated at 37° C. for 2 hours followed by β-catenin detection as described. For example, the combination of peptides 4, 5, and 6 (SEQ ID NOS; 4, 5 and 6) shows a dramatic decrease in the ALEXA FLUOR® 488 emission and resulted in less than 10% β-catenin bound. The SW480 lysate standard curve was generated by varying the concentration of lysate without any peptides present.

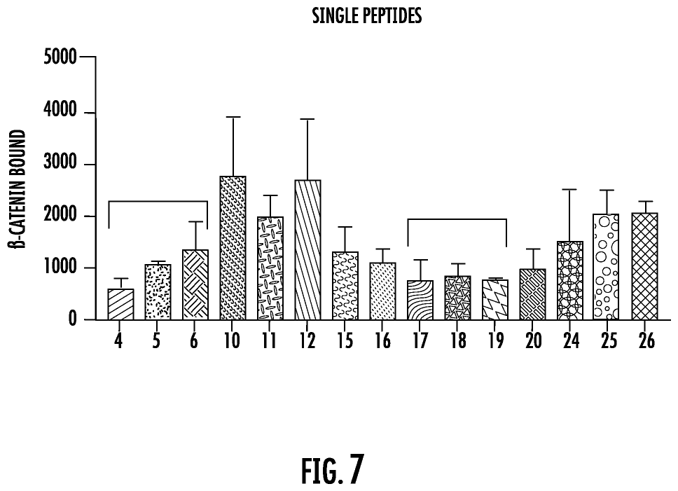

In a screening of individual peptides with the sandwich ELISA assay, peptides 4, 5, 6, 17, 18, and 19 (SEQ ID NOs: 4, 5, 6, 17, 18, and 19) showed efficacy in disrupting the interaction between TBL1 and β-catenin (see FIG. 7), suggesting that combinations of these peptides could provide effective therapeutic agents in the treatment of cancer.

### Example 3. Further Screening of Peptide Library for In Vitro Activity

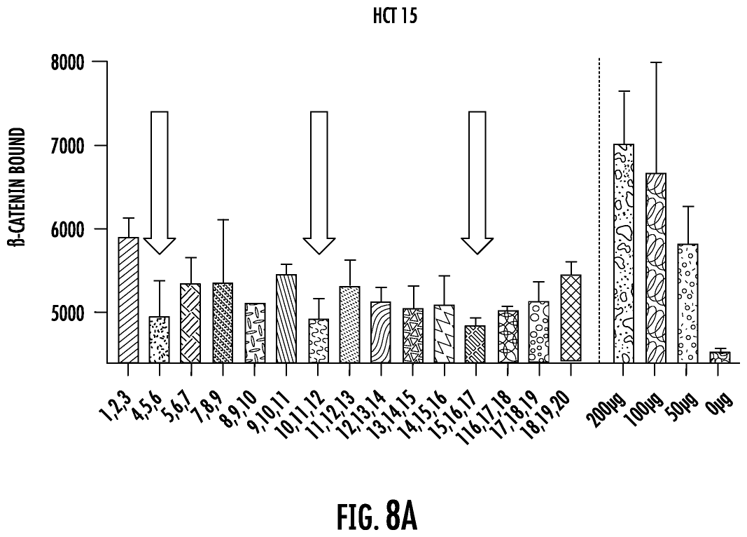

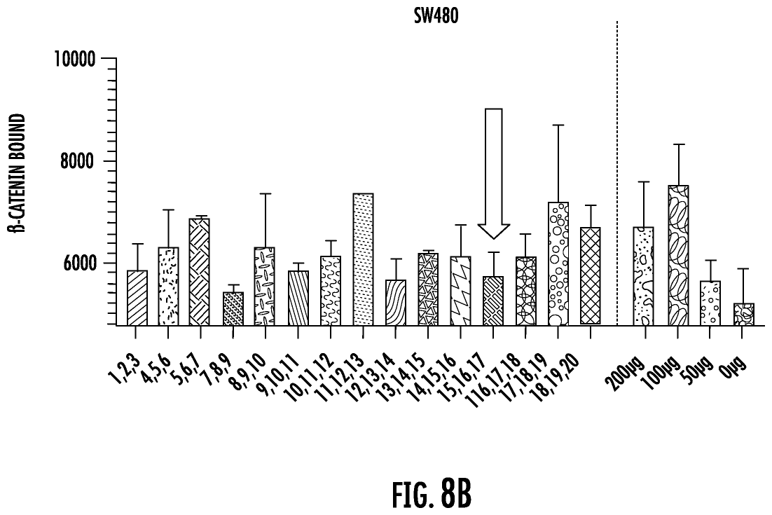

Additional screening was performed with the peptide library using the assay described in Example 2 with the colon cancer cell lines HCT15 and SW480. Combinations of three peptides were evaluated as indicated in FIGS. 8A and 8B. This additional screening confirmed the activities of the following combinations of peptides: (1) 4, 5, and 6; (2) 10, 11, and 12; and (3) 15, 16, and 17 (see FIGS. 8A and 8B).

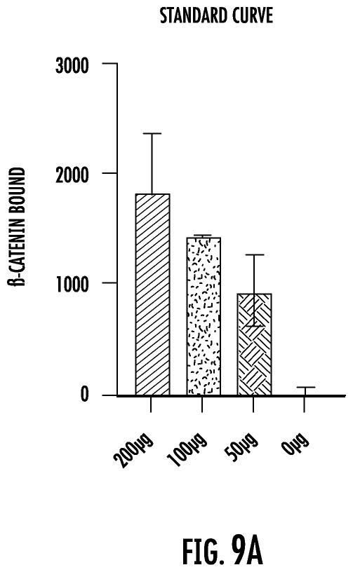

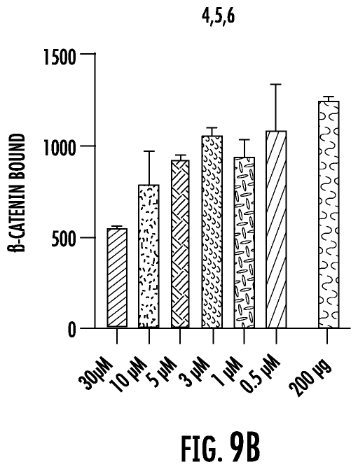

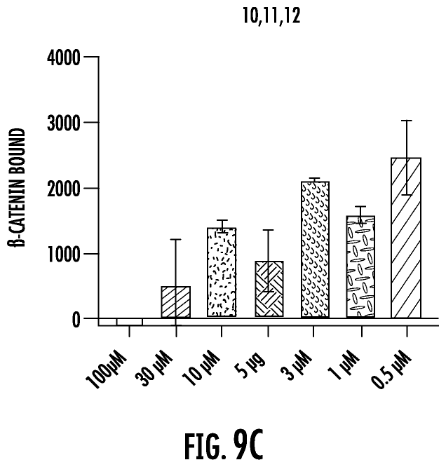

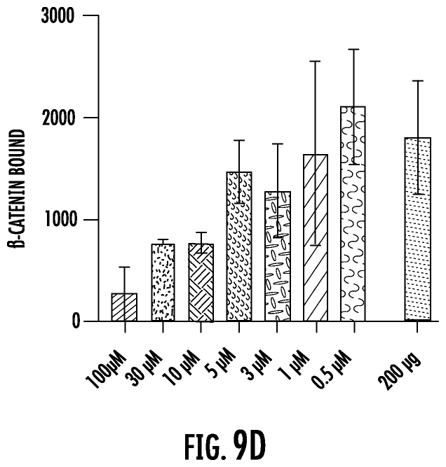

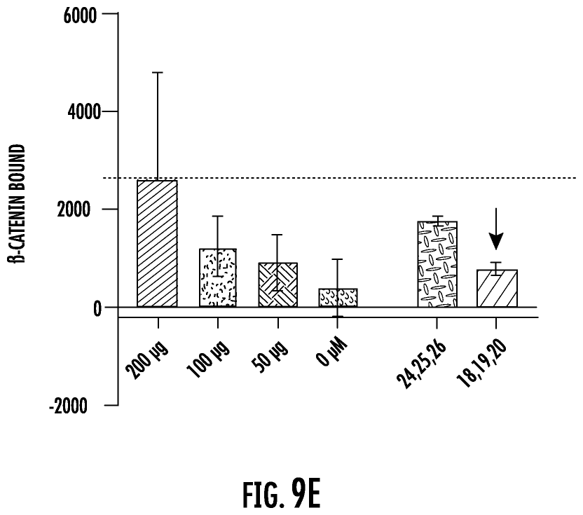

Combinations of three peptides were subsequently assayed at varying concentrations to show a dose response in the disruption of the interaction between TBL1 and β-catenin. These dose response assays demonstrated the efficacies of the following combinations of peptides: (1) 4, 5, and 6; (2) 10, 11, and 12; and (3) 18, 19, and 20 (see FIGS. 9A to 9D). A further assay demonstrated the efficacy of the following combinations of peptides: (1) 18, 19, and 20; (2) 24, 25, and 26 (see FIG. 9E).

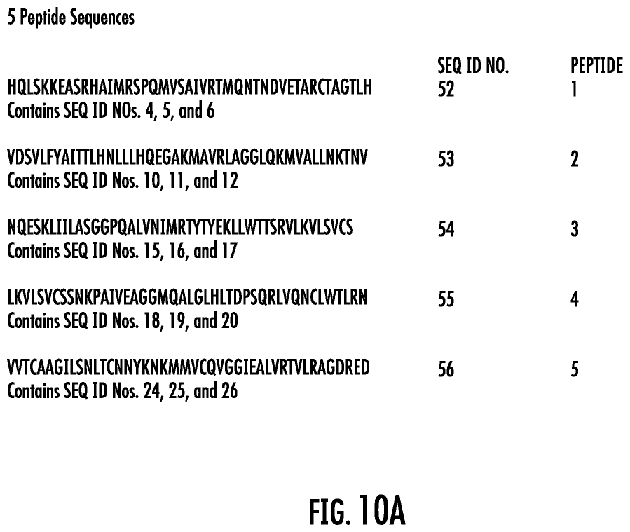

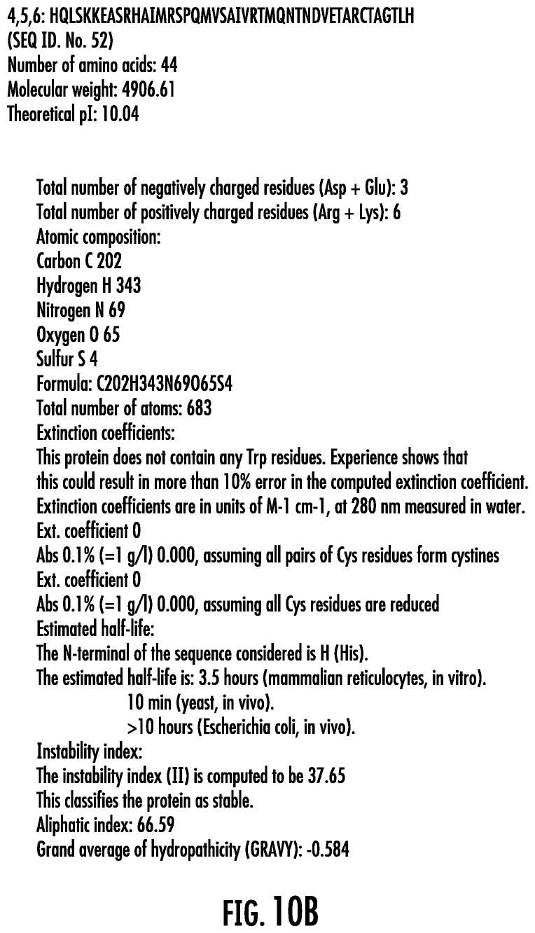

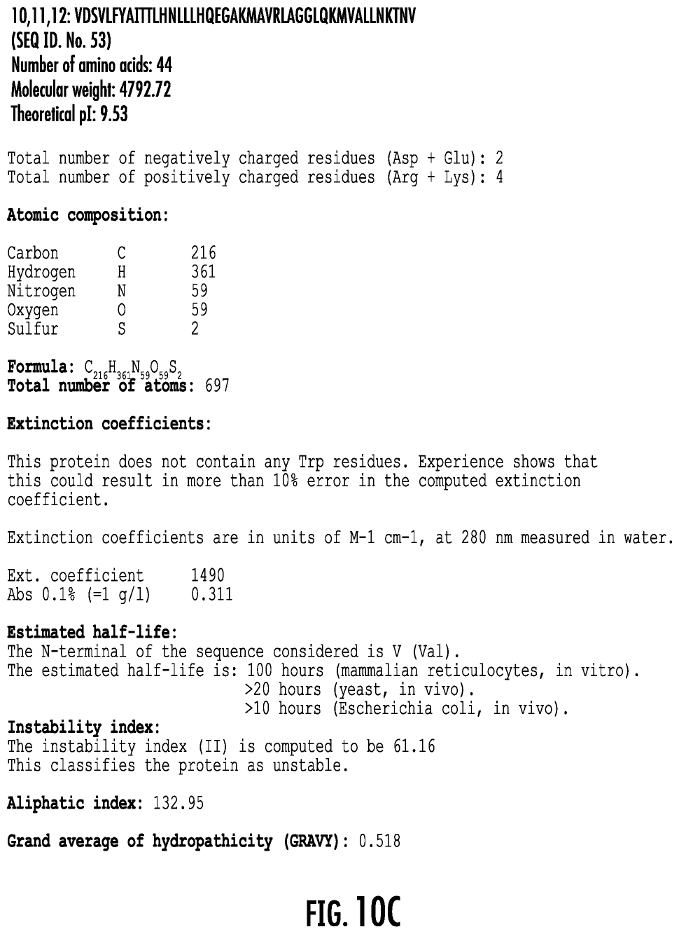

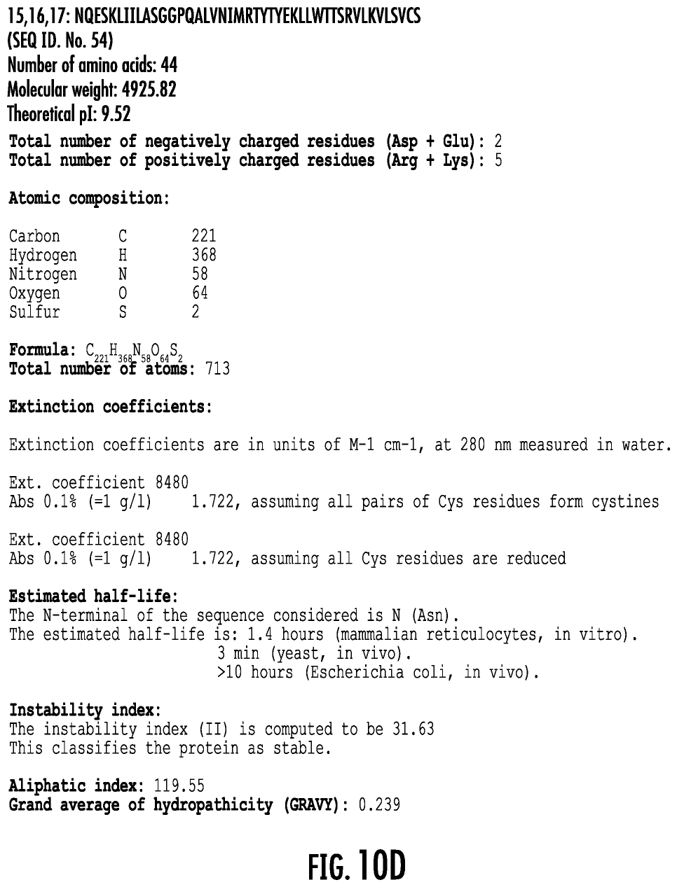

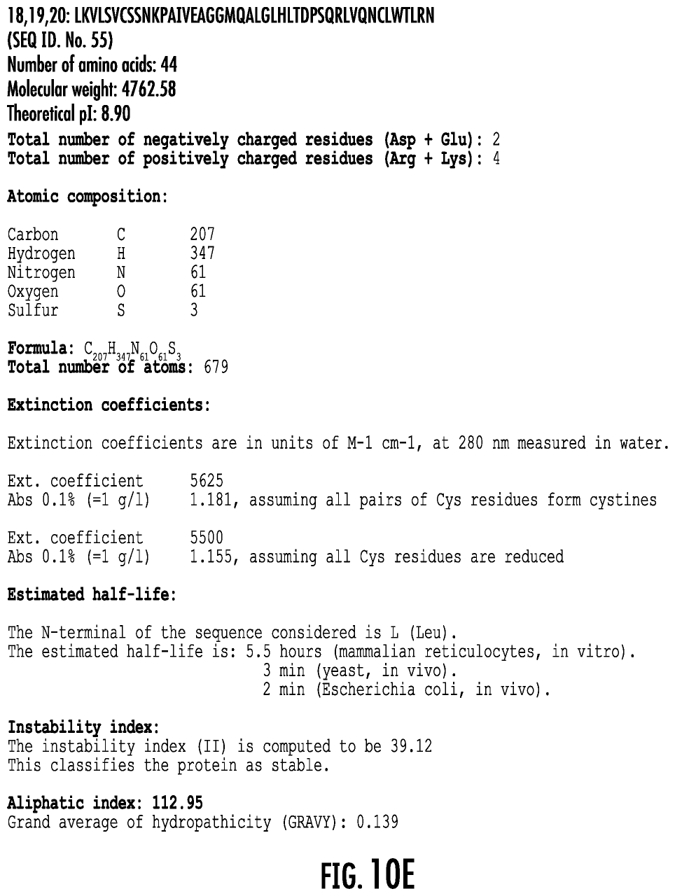

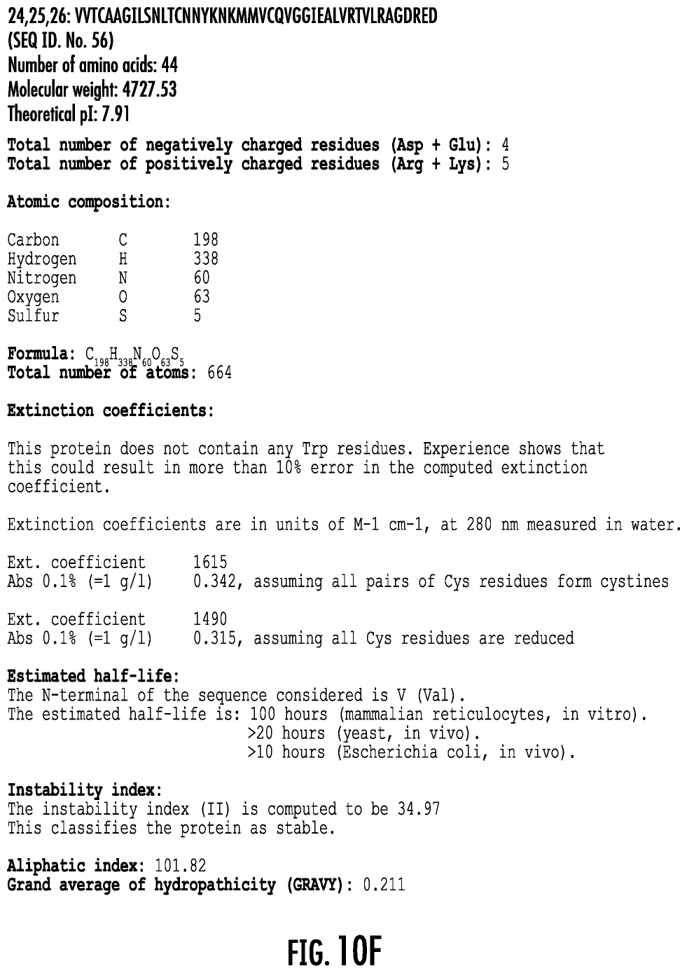

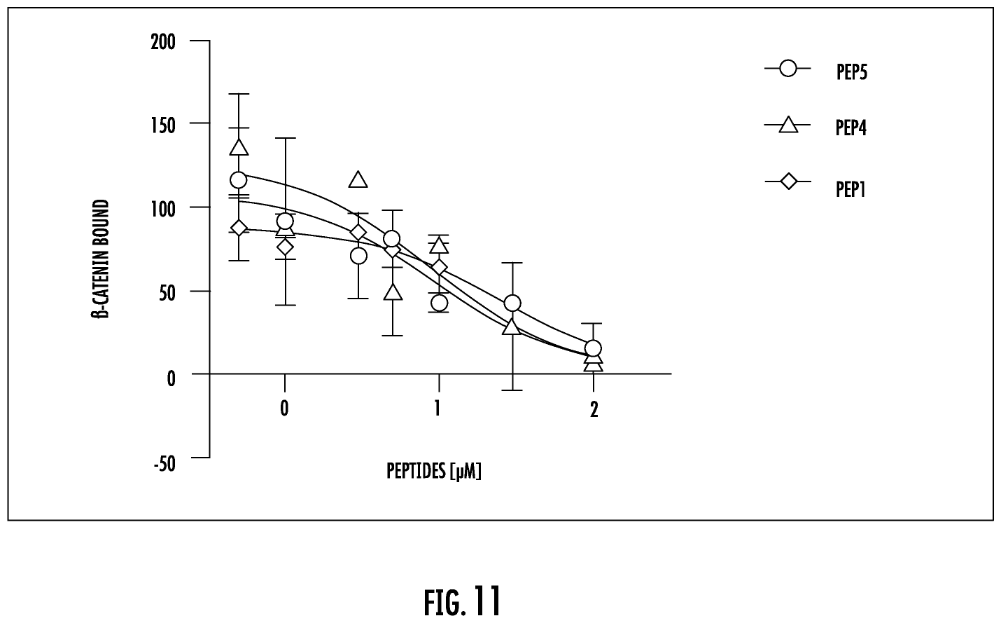

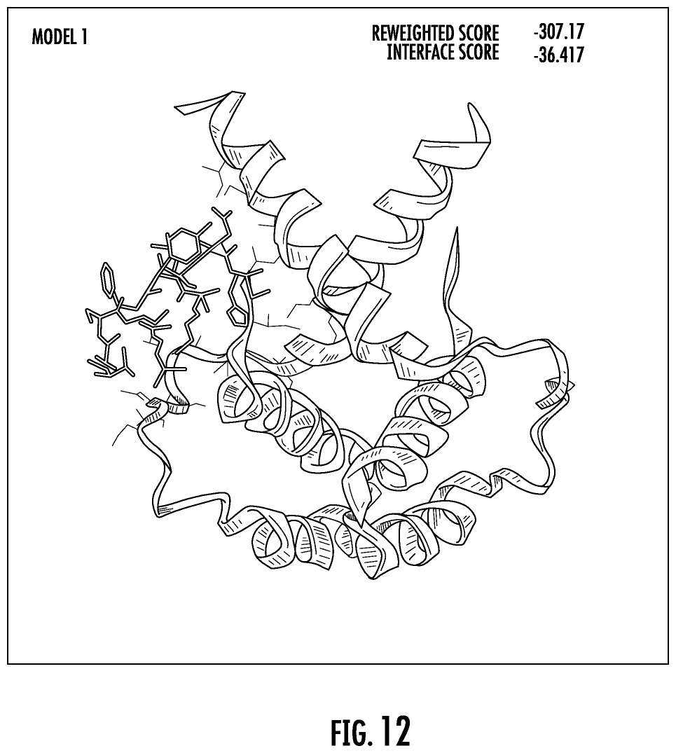

Based on the results of peptide screening, five longer peptides were selected for further evaluation (i.e., Peptides 1-5; SEQ ID NOs: 52-56 respectively; see FIGS. 10A to 10F). Peptide 1 (“PEP1”), Peptide 4 (“PEP4), and Peptide 5 (“PEP5”) were evaluated in a homologous competitive ELISA assay performed with increasing logarithmic concentrations of each peptide. Each of the three selected peptides were able to displace β-catenin from the TBL1 complex in a dose dependent manner (see FIG. 11). A three-dimensional model was generated with selected peptides to identify putative binding sites on the TBL1 complex (see FIG. 12).

SEQ ID NOs. 52-56 are as follows.

SEQ ID NO. 52:
HQLSKKEASRHAIMRSPQMVSAIVRTMONTNDVETARCTAGTLH
SEQ ID NO. 53:
VDSVLFYAITTLHNLLLHQEGAKMAVRLAGGLQKMVALLNKTNV
SEQ ID NO. 54:
NQESKLIILASGGPQALVNIMRTYTYEKLLWTTSRVLKVLSVCS
SEQ ID NO. 55:
LKVLSVCSSNKPAIVEAGGMQALGLHLTDPSQRLVQNCLWTLRN
SEQ ID NO. 56:
VVTCAAGILSNLTCNNYKNKMMVCQVGGIEALVRTVLRAGDRED

### Example 4. Pull-Down Experiment with SW480 Cells Transfected with Peptides

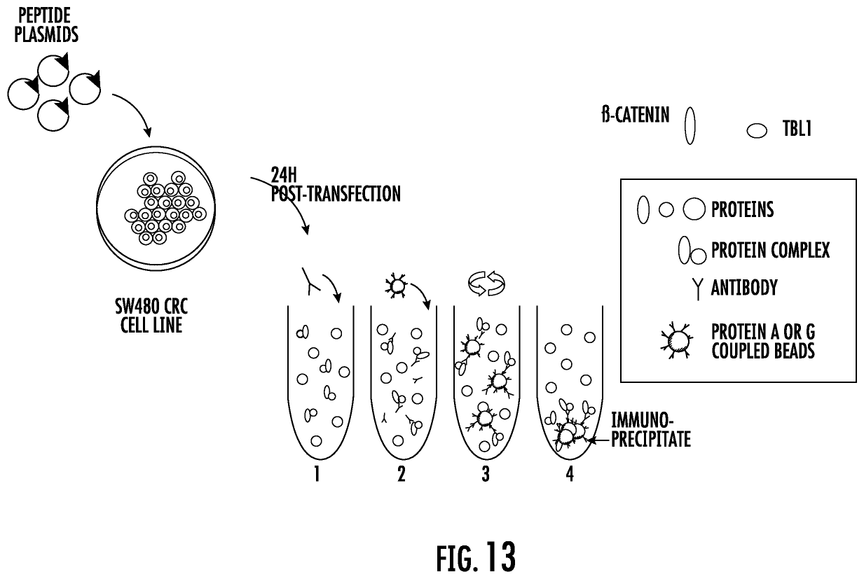

Pull-down experiments using lysates of SW480 cells transfected with Peptides 1-5 were conducted as outlined in FIG. 13. Tegatrabetan (BC2059) is an antagonist of β-Catenin and was used as a positive control in the pull-down experiments. Tegatrabetan (BC2059) treatment disrupts the binding of β-catenin with the scaffold protein transducin β-like 1 (TBL1) leading to proteasomal degradation of β-catenin and a decline in the nuclear levels of β-catenin. Green fluorescent protein (GFP) was also transfected into the SW480 cells as a negative control.

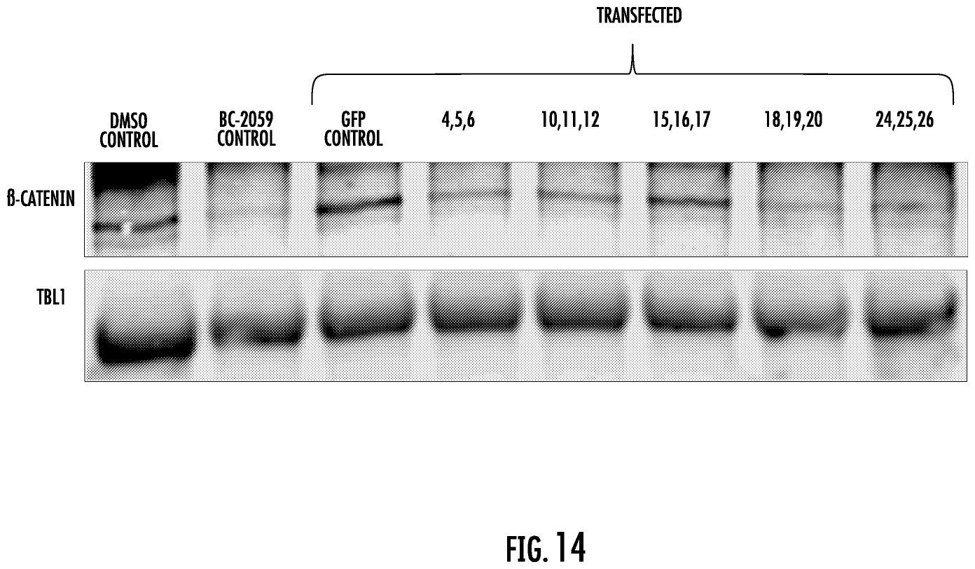

Each of the peptides decreased the amount of β-catenin bound to TBL1 in the transfected SW480 cells with the most pronounced effects observed with Peptides 1, 2, 4, and 5 (see FIG. 14).

### Example 5. TOPFlash TCF/LEF Reporter Assay with Peptides

A TOPFlash TCF/LEF reporter assay was performed with Peptides 1-5 as described in Veeman M T, et al. (2003) Curr Biol. 13(8):680-5. This is a luciferase reporter assay of (3-catenin-mediated transcriptional activation. Tegatrabetan (BC2059) was used as a positive control in the TOPFlash TCF/LEF reporter assay. Briefly, TCF/LEF reporter cells were transfected with the nucleofection kit. After 48 hours, the ONE-STEP™ luciferase assay was performed.

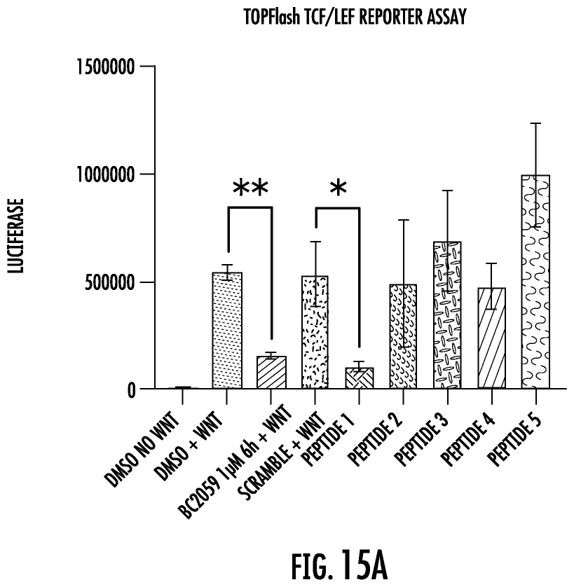

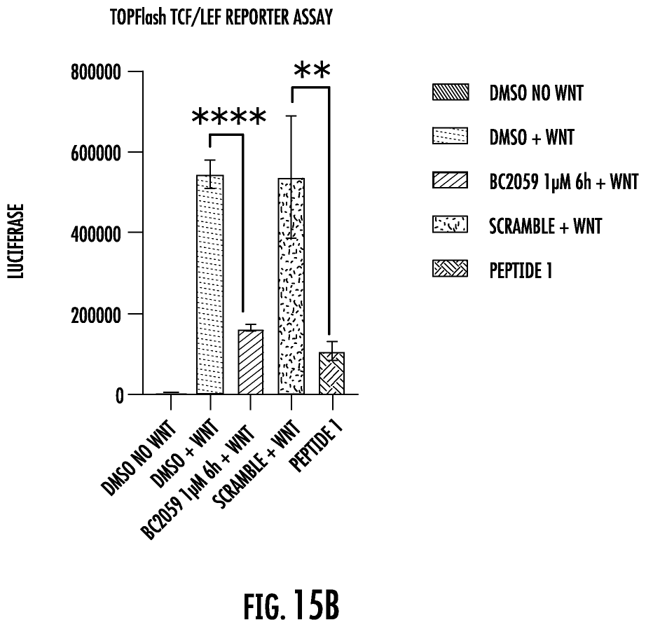

In the initial evaluation of all five peptides, Peptide 1 produced a significant decrease in β-catenin-mediated transcriptional activation (see FIG. 15A). This was demonstrated by decreased levels of luciferase indicating a decrease in TCF/LEF activation and downstream transcription for peptide 1. A second experiment evaluating only Peptide 1 confirmed the earlier result (see FIG. 15B).

### Example 6. ELISA Assay with Dansyl-Tagged Peptides and TBL1

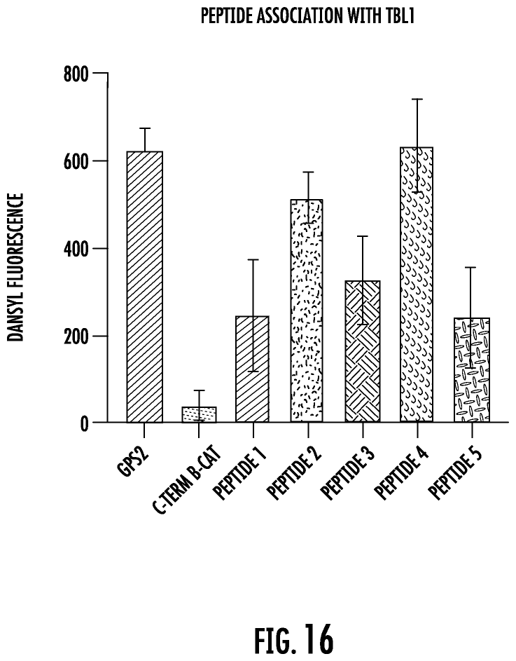

An ELISA assay using dansyl-tagged peptides confirmed that Peptides 1-5 associate with TBL1. High levels of dansyl fluorescence indicated peptide binding to TBL1 protein (see FIG. 16). GPS2 is known to interact with TBL1 and was the positive control while the C-terminus of β-catenin is known to not bind to TBL1 and was the negative control (see FIG. 16).

### Example 7. Evaluation of Peptides with “Nano-Ghost” Technology

The peptides that effectively disrupt TBL1:$-catenin association and knock down $-catenin in vitro will be tested in vivo through the “nano-ghosts” platform. This highly innovative, immune-evasive “nano-ghost” technology allows delivery of proteins effectively to cancer cells. See Oieni, J., et al., (2021) Journal of Controlled Release: Official Journal of the Controlled Release Society, 333: 28-40. The peptides will be loaded into the “nano-ghosts” and subsequently tested for efficacy in treating cancer.

### Example 8. Evaluation with Sandwich ELISA Approach

To identify the peptide or peptides able to disrupt the interaction between TBL1 and (3-catenin, we tested the Sandwich ELISA approach. In this assay TBL1/β-catenin complex from lysates of colon cancer cell with active Wnt signaling, is captured in Protein G ELISA 96 well plate coated with antibody that recognizes TBL1. To ensure activation of Wnt signaling, the cells are pre-treated with 20 mM LiCl for 12 h a 37° C. The bound complex is then exposed to peptides from the library either as single peptide or in a combination of 2 or 3 peptides to ensure coverage of the potential binding site between TBL1 and β-catenin. After washing away unbound proteins, the plates are incubated with β-catenin antibodies labeled with Alexa Fluor 488 dye, and the level of β-catenin associated to TBL1 is quantified by a plate reader that detects the 488 fluorescence probe. As a font of TBL1/β-catenin complex we used colon cancer cell lines HCT15 and SW480. All the experiments are conducted in triplicated to ensure statistical significance, and to reduce variability we perform the assays by using the EpiMotion liquid handler. We designed a program that controls the peptide seeding into the ELISA plate wells: 1, 2 or 3 peptides per well, with overlap of one peptides in each well.

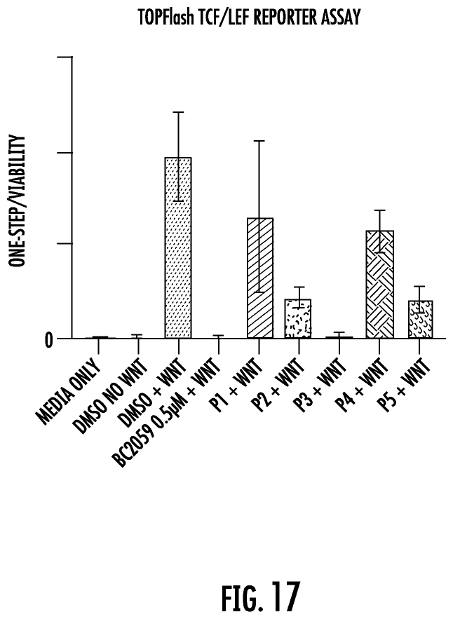

FIG. 17 presents a TOPFlash assay to determine transcription activity downstream TCF/LEF: HEK293 cells containing a luciferase downstream TCF/LEF were treated with LiCi 16 hours prior to treatment to stimulate the Wnt pathway. Then they were treated with recombinant Wnt3a to stimulate transcription and peptide at 5 μM or BC-2059 at 0.5 μM for 6 hours before luciferase expression was quantified with the One-Step Luciferase assay system. We identified that peptides 2, 3, and 5 all have decreased levels of luciferase after treatment identifying that these peptides disrupt D-catenin's activity as a transcriptional coactivator.

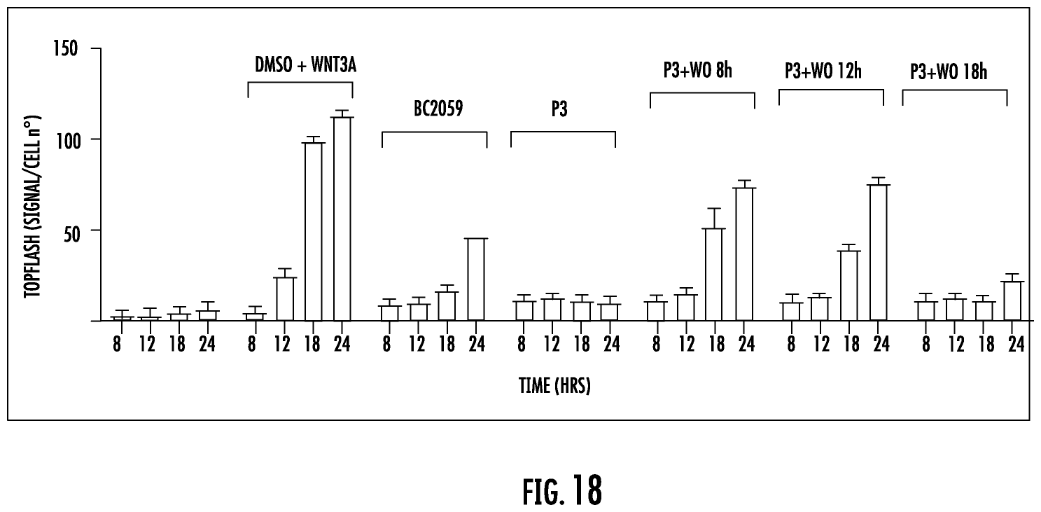

FIG. 18 presents wash out TOPFlash at 8, 12,18, and 24 hour time points: to investigate the effects of peptide 3 over time with a washout TOPFlash assay. To do this we followed the same procedure previously stated but performed washouts at 8, 12, 18, and 24 hour time points.

This experiment was completed in duplicate. We identified that if the peptide is washed out after 8 and 12 hours, we see an increase in luciferase produced compared to the non washout control. This identifies that peptide 3 is specific and effective at blocking transcription downstream TCF/LEF.

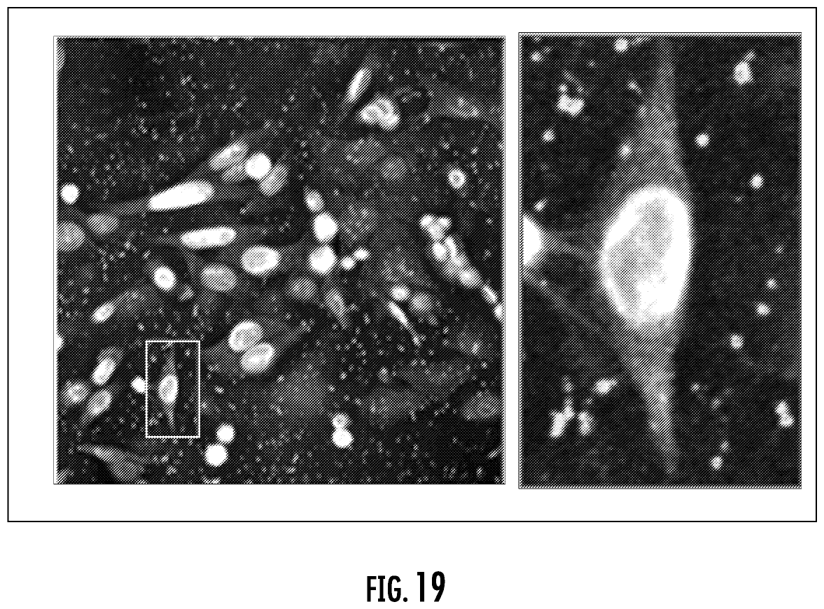

FIG. 19 shows immunofluorescence images taken to identify if CPP peptides penetrate SW480 cells. SW480 cells were seeded onto glass slides then treated with 5 μM peptide 4 and incubated for 6 hours. After incubation the cells were washed and fixed then treated with Flag antibody overnight. After this a green fluorescent a secondary antibody was incubated. The cells were washed and mounted with DAPI mounting media then visualized on a confocal microscope. CPP-peptides do penetrate the cells, however they appear to form aggregates which we hypothesize is due to instability of peptide structure.

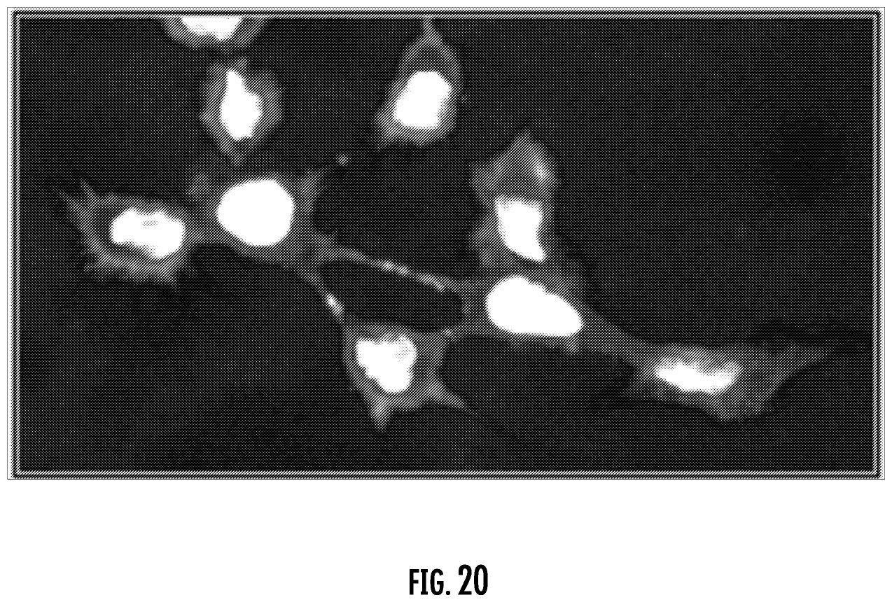

In FIG. 20, to identify the localization of these peptides into SW480 cells we repeated the immunofluorescence as previously described but added and additional actin stain to aid in identification of peptide localization. These peptides were also incubated for 24 hours before fixation to allow more time to penetrate. We found that the peptides are found within the cells however they appear to cluster in the cytoplasmic extensions.

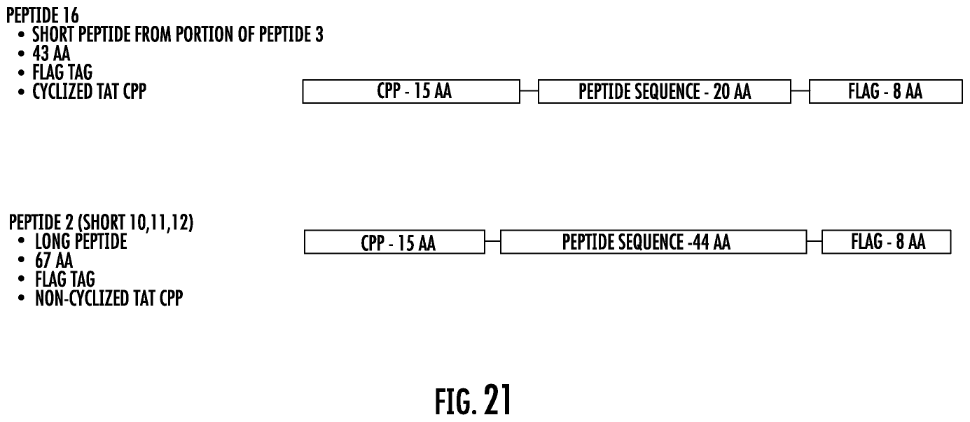

FIG. 21 presents certain peptide specifications. Due to the concerns of our peptide stabilization, we have optimized the peptides to be shorter and to have a cyclized CPP. We believe that these modifications will aid in the peptide stability and will make our peptides more effective.

Additional peptides short peptides (10 aa+CPP) include:

aa N-A:
(SEQ ID NO. 57)
CGRKKRRQRRRPPQCNQESKLIILA
aa L-Q:
(SEQ ID NO. 58)
CGRKKRRQRRRPPQCLIILASGGPQ
aa S-I:
(SEQ ID NO. 59)
CGRKKRRQRRRPPQCSGGPQALVNI
aa A-T:
(SEQ ID NO. 60)
CGRKKRRQRRRPPQCALVNIMRTYT
aa M-L:
(SEQ ID NO. 61)
CGRKKRRQRRRPPQCMRTYTYEKLL
aa Y-R:
(SEQ ID NO. 62)
CGRKKRRQRRRPPQCYEKLLWTTSR
aa W-L:
(SEQ ID NO. 63)
CGRKKRRQRRRPPQCWTTSRVLKVL
aa V-S:
(SEQ ID NO. 64)
CGRKKRRQRRRPPQCVLKVLSVASS
Scramble:
(SEQ ID NO. 65)
CGRKKRRQRRRPPQCAEISLIQKLN

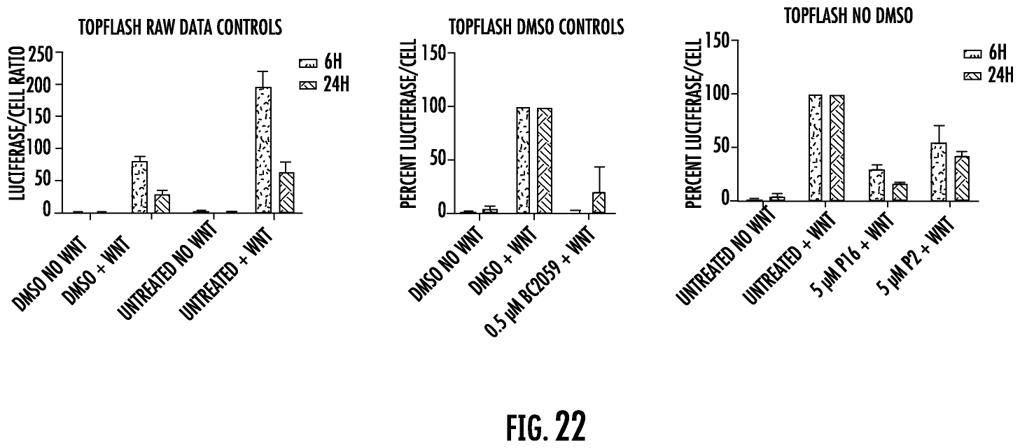

FIG. 22 presents TOPFlash assay to determine transcription activity downstream TCF/LEF: HEK293 cells containing a luciferase downstream TCF/LEF were with LiCl 16 hours prior to treatment to stimulate the Wnt pathway. Then they were treated with recombinant Wnt3a to stimulate transcription and peptide 16 (short) and 2 (long) at 5 μM or BC-2059 at 0.5 μM for 6 hours before luciferase expression was quantified with the One-Step Luciferase assay system. Peptides 16 showed higher efficacy to promote decreased levels of luciferase after treatment compared to peptide 2, though the difference is not statistically significant.

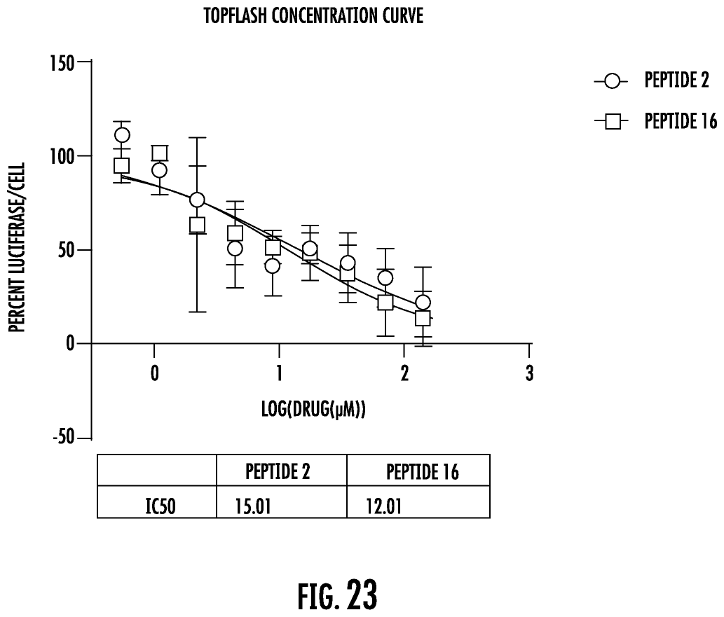

FIG. 23 presents TOPFlash assay results to determine transcription activity downstream TCF/LEF dependency to peptides: HEK293 cells containing a luciferase downstream TCF/LEF were with LiCl 16 hours prior to treatment to stimulate the Wnt pathway. Then they were treated with recombinant Wnt3a to stimulate transcription and peptide 16 (short) and 2 (long) at different concentrations (300 M-0.01 μM) for 6 hours before luciferase expression was quantified with the One-Step Luciferase assay system. TCF/LEF transcriptional activity decreases with treatment with increased concentration of peptides 2 and 16.

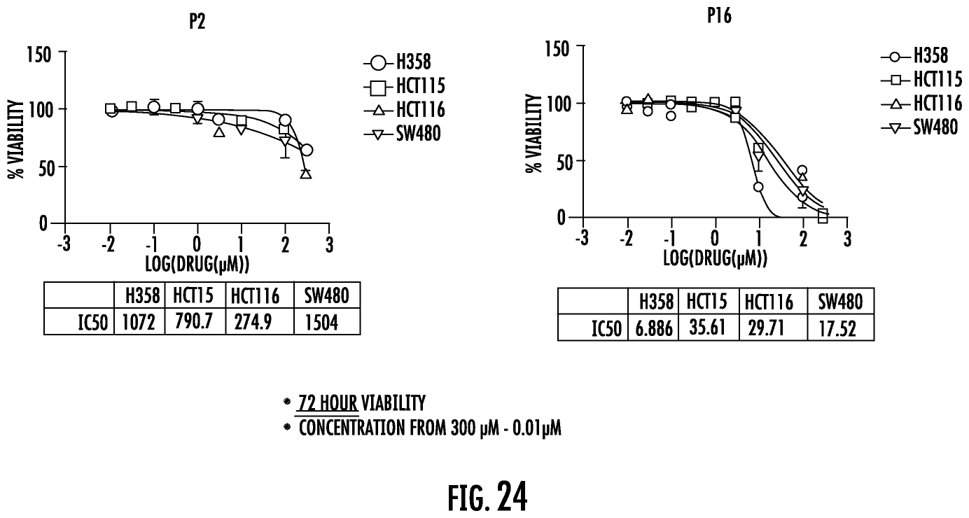

FIG. 24 shows the cytotoxic effect of peptides 2 and 6 on different cell lines (H358 (lung cancer) HCT15, HCT116, and SW480 (colon cancer) characterized by WNT pathway activation/dependency. Cells were incubated with increasing concentrations of peptide 16 (short) and 2 (long) at different concentrations (300 μM-0.01 μM) for 72 hours. Cell viability was assessed by CellTiter Glo.

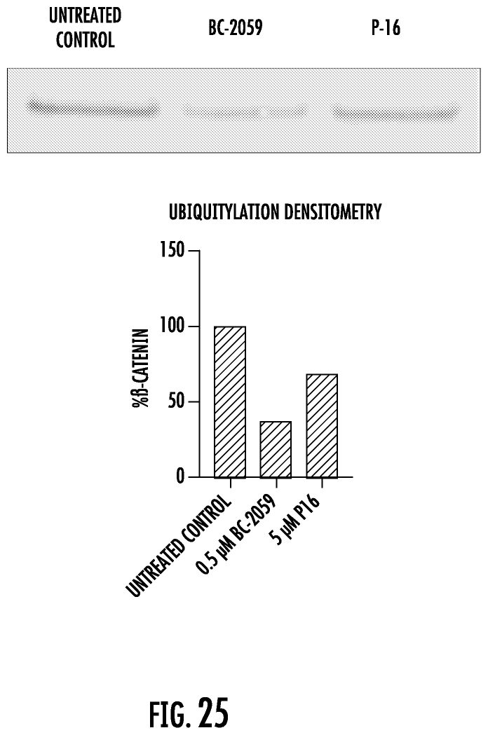

FIG. 25 shows peptide 16 promotes polyubiquitination of β-catenin. HCT 15 cells were treated with 20 mM LiCl, the universal activator of Wnt signaling, in presence/absence of 0.5 μM BC-2059 or 5 μM peptide 16 for 6 hours. Harvested cells were subjected to lysis and samples were processed for enrichment of polyubiquitinated protein conjugates with the Ubiquitin Enrichment Kit (Thermo Scientific) according to the manufacturer's protocol. Samples were resolved in 4-12% SDS-PAGE (Invitrogen). Western blot analysis was performed using β-catenin antibody (BD Biosciences) and Odyssey Imager (Li-Cor Biosciences).The results show that peptide 16 promotes polyubiquitination of β-catenin.

### CLAUSES

Clause 1. A method of treating cancer in a subject, the method comprising:
   administering to the subject a therapeutically effective amount of a pharmaceutical composition comprising a first peptide, wherein the first peptide comprises a first amino acid sequence selected from the group consisting of SEQ ID NOs: 1-33, 52-65, and function-conservative variants thereof.
Clause 2. The method of clause 1, wherein the pharmaceutical composition further comprises a second peptide having a second amino acid sequence selected from the group consisting of SEQ ID NOs: 1-33, 52-65, and function-conservative variants thereof;
   with the proviso that the first peptide and the second peptide do not comprise the same amino acid sequence.
Clause 3. The method of clause 2, wherein the pharmaceutical composition further comprises a third peptide having a third amino acid sequence selected from the group consisting of SEQ ID NOs: 1-33, 52-65, and function-conservative variants thereof;
   with the proviso that the first peptide, the second peptide, and the third peptide do not comprise the same amino acid sequence.
Clause 4. The method of any one of clauses 1-3, wherein the first peptide, the second peptide, and the third peptide are each less than 50 amino acids in length.
Clause 5. The method of any of one of clauses 1-4, wherein the function-conservative variants individually consist of a sequence that differs from any one of SEQ ID NOs: 1-33 and 52-65 by 1, 2, 3, 4, or 5 amino acids.
Clause 6. The method of any one of clauses 1-5, wherein the first peptide, the second peptide, and the third peptide individually comprise an amino acid sequence selected from the group consisting of SEQ ID NOs: 52-56.
Clause 7. The method of any one of clauses 1-6, wherein the first peptide comprises an amino acid sequence of SEQ ID NO: 52.
Clause 8. The method of any one of clauses 1-7, wherein the pharmaceutical composition inhibits beta-catenin (β-catenin) association with Transducin Beta-like protein 1 (TBL1) to disrupt formation of a β-catenin:TBL1 complex in the subject.
Clause 9. The method of any one of clauses 1-8, wherein the cancer is a Wnt/β-catenin active cancer.
Clause 10. The method of clause 9, further comprising determining that the subject has a Wnt/β-catenin active cancer.
Clause 11. The method of any one of clauses 1-10, wherein the cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer.
Clause 12. The method of any one of clauses 1-11, wherein the subject is human.
Clause 13. The method of any one of clauses 1-12, wherein administering the pharmaceutical composition to the subject is selected from the group consisting of intranasal administration, inhalational administration, intravenous administration, oral administration, and parenteral administration.
Clause 14. The method of any one of clauses 1-13, wherein the pharmaceutical composition comprises one or more pharmaceutically acceptable excipients.
Clause 15. A method of treating a subject having a disease, the method comprising the step of
   administering to the subject a therapeutically effective amount of a pharmaceutical composition comprising a first peptide having at least 80% homology to a first amino acid sequence selected from the group consisting of SEQ ID NOs: 1-33 and 52-65.
Clause 16. The method of clause 15, wherein the first peptide has at least 90% homology to the first amino acid sequence selected from the group consisting of SEQ ID NOs: 1-33 and 52-65.
Clause 17. The method of clause 16, wherein the first peptide has at least 95% homology to the first amino acid sequence selected from the group consisting of SEQ ID NOs: 1-33 and 52-65.
Clause 18. The method of any one of clauses 15-17, wherein the pharmaceutical composition further comprises a second peptide having at least 80% homology to a second amino acid sequence selected from the group consisting of SEQ ID NOs: 1-33 and 52-65, or at least 90% homology to the second amino acid sequence selected from the group consisting of SEQ ID NOs: 1-33 and 52-65, or at least 95% homology to the second amino acid sequence selected from the group consisting of SEQ ID NOs: 1-33 and 52-65;
   with the proviso that the first peptide and the second peptide do not comprise the same amino acid sequence.
Clause 19. The method of clause 18, wherein the pharmaceutical composition further comprises a third peptide having at least 80% homology to a third amino acid sequence selected from the group consisting of SEQ ID NOs: 1-33 and 52-65, or at least 90% homology to the third amino acid sequence selected from the group consisting of SEQ ID NOs: 1-33 and 52-65, or at least 95% homology to the third amino acid sequence selected from the group consisting of SEQ ID NOs: 1-33 and 52-65;
   with the proviso that the first peptide, the second peptide, and the third peptide do not comprise the same amino acid sequence.
Clause 20. The method of any one of clauses 15-19, wherein the first peptide, the second peptide, and the third peptide individually comprise an amino acid sequence having at least 80% homology to sequence selected from the group consisting of SEQ ID NOs: 52-56.
Clause 21. The method of any one of clauses 15-20, wherein the first peptide comprises an amino acid sequence having at least 80% homology to SEQ ID NO: 52.
Clause 22. The method of any one of clauses 15-21, wherein the pharmaceutical composition inhibits beta-catenin (β-catenin) association with Transducin Beta-like protein 1 (TBL1) to disrupt formation of a β-catenin:TBL1 complex in the subject.
Clause 23. The method of any one of clauses 15-22, wherein the pharmaceutical composition comprises one or more pharmaceutically acceptable excipients.
Clause 24. The method of any one of clauses 15-23, wherein administering the pharmaceutical composition to the subject is selected from the group consisting of intranasal administration, inhalational administration, intravenous administration, oral administration, and parenteral administration.
Clause 25. The method of any of clauses 15-24, wherein the subject is human.
Clause 26. The method of any of clauses 15-25, wherein the disease is a Wnt/β-catenin active cancer.
Clause 27. The method of clause 26, further comprising determining that the subject has a Wnt/D-catenin active cancer.
Clause 28. The method of clause 26 or 27, wherein the Wnt/β-catenin active cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer.
Clause 29. A pharmaceutical composition for the treatment of cancer, the pharmaceutical composition comprising:
   a first peptide, wherein the first peptide comprises a first amino acid sequence selected from the group consisting of SEQ ID NOs: 1-33, 52-65, and function-conservative variants thereof.
Clause 30. The pharmaceutical composition of clause 29, further comprising a second peptide, wherein the second peptide comprises a second amino acid sequence selected from the group consisting of SEQ ID NOs: 1-33, 52-65, and function-conservative variants thereof,
   with the proviso that the first peptide and the second peptide do not comprise the same amino acid sequence.
Clause 31. The pharmaceutical composition of clause 30, further comprising a third peptide, wherein the third peptide comprises a third amino acid sequence selected from the group consisting of SEQ ID NOs: 1-33, 52-65, and function-conservative variants thereof;
   with the proviso that the first peptide, the second peptide, and the third peptide do not comprise the same amino acid sequence.
Clause 32. The pharmaceutical composition of any of clauses 29-31, further comprising at least one pharmaceutically acceptable carrier.
Clause 33. The pharmaceutical composition of any one of clauses 29-32, wherein the first peptide, the second peptide, and the third peptide are each less than 50 amino acids in length.
Clause 34. The pharmaceutical composition of any one of clauses 29-33, wherein the pharmaceutical composition is designed for intranasal administration, inhalational administration, intravenous administration, oral administration, or parenteral administration.
Clause 35. The pharmaceutical composition of any one of clauses 29-34, wherein the disease is a Wnt/β-catenin active cancer.
Clause 36. The pharmaceutical composition of any one of clauses 29-35, wherein the Wnt/β-catenin active cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. Stamos J L, Weis W I. The beta-catenin destruction complex. Cold Spring Harb Perspect Biol 2013;5(1):a007898 doi 10.1101/cshperspect.a007898.
2. Choi H K, Choi K C, Yoo J Y, Song M, Ko S J, Kim C H, et al. Reversible SUMOylation of TBL1-TBLR1 regulates beta-catenin-mediated Wnt signaling. Mol Cell 2011; 43(2):203-16 doi 10.1016/j.molcel.2011.05.027.
3. Dimitrova Y N, Li J, Lee Y T, Rios-Esteves J, Friedman D B, Choi H J, et al. Direct ubiquitination of beta-catenin by Siah-1 and regulation by the exchange factor TBL1. J Biol Chem 2010; 285(18):13507-16 doi 10.1074/jbc.M109.049411.
4. Li J, Wang C Y. TBL1-TBLR1 and beta-catenin recruit each other to Wnt target-gene promoter for transcription activation and oncogenesis. Nat Cell Biol 2008; 10(2):160-9 doi 10.1038/ncb1684.

1. A method of treating cancer in a subject, the method comprising:
administering to the subject a therapeutically effective amount of a pharmaceutical composition comprising a first peptide, wherein the first peptide comprises a first amino acid sequence selected from the group consisting of SEQ ID NOs: 1-33, 52-65, and function-conservative variants thereof.

2. The method of claim 1, wherein the pharmaceutical composition further comprises a second peptide having a second amino acid sequence selected from the group consisting of SEQ ID NOs: 1-33, 52-65, and function-conservative variants thereof;
with the proviso that the first peptide and the second peptide do not comprise the same amino acid sequence.

3. The method of claim 2, wherein the pharmaceutical composition further comprises a third peptide having a third amino acid sequence selected from the group consisting of SEQ ID NOs: 1-33, 52-65, and function-conservative variants thereof;
with the proviso that the first peptide, the second peptide, and the third peptide do not comprise the same amino acid sequence.

4. The method of claim 3, wherein
the first peptide, the second peptide, and the third peptide are each less than 50 amino acids in length; and/or
wherein the first peptide, the second peptide, and the third peptide individually comprise an amino acid sequence selected from the group consisting of SEQ ID NOs: 52-56.

5. The method of claim 1, wherein the function-conservative variants individually consist of a sequence that differs from any one of SEQ ID NOs: 1-33 and 52-65 by 1, 2, 3, 4, or 5 amino acids; and/or
wherein the first peptide comprises an amino acid sequence of SEQ ID NO: 52; and/or
wherein the pharmaceutical composition inhibits beta-catenin (β-catenin) association with Transducin Beta-like protein 1 (TBL1) to disrupt formation of a β-catenin:TBL1 complex in the subject.

6-8. (canceled)

9. The method of claim 1, wherein the cancer is a Wnt/β-catenin active cancer; and/or
wherein the cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer.

10. The method of claim 9, further comprising determining that the subject has a Wnt/β-catenin active cancer.

11. (canceled)

12. The method of claim 1, wherein the subject is human.

13. The method of claim 1, wherein administering the pharmaceutical composition to the subject is selected from the group consisting of intranasal administration, inhalational administration, intravenous administration, oral administration, and parenteral administration; and/or
wherein the pharmaceutical composition comprises one or more pharmaceutically acceptable excipients.

14. (canceled)

15. A method of treating a subject having a disease, the method comprising the step of:
administering to the subject a therapeutically effective amount of a pharmaceutical composition comprising a first peptide having at least 80% homology to a first amino acid sequence selected from the group consisting of SEQ ID NOs: 1-33 and 52-65; or
wherein the first peptide has at least 90% homology to the first amino acid sequence selected from the group consisting of SEQ ID NOs: 1-33 and 52-65; or
wherein the first peptide has at least 95% homology to the first amino acid sequence selected from the group consisting of SEQ ID NOs: 1-33 and 52-65; or
wherein the first peptide comprises an amino acid sequence having at least 80% homology to SEQ ID NO: 52.

16-17. (canceled)

18. The method of claim 15, wherein the pharmaceutical composition further comprises a second peptide having at least 80% homology to a second amino acid sequence selected from the group consisting of SEQ ID NOs: 1-33 and 52-65, or at least 90% homology to the second amino acid sequence selected from the group consisting of SEQ ID NOs: 1-33 and 52-65, or at least 95% homology to the second amino acid sequence selected from the group consisting of SEQ ID NOs: 1-33 and 52-65;
with the proviso that the first peptide and the second peptide do not comprise the same amino acid sequence.

19. The method of claim 18, wherein the pharmaceutical composition further comprises a third peptide having at least 80% homology to a third amino acid sequence selected from the group consisting of SEQ ID NOs: 1-33 and 52-65, or at least 90% homology to the third amino acid sequence selected from the group consisting of SEQ ID NOs: 1-33 and 52-65, or at least 95% homology to the third amino acid sequence selected from the group consisting of SEQ ID NOs: 1-33 and 52-65;
with the proviso that the first peptide, the second peptide, and the third peptide do not comprise the same amino acid sequence.

20. The method of claim 19, wherein the first peptide, the second peptide, and the third peptide individually comprise an amino acid sequence having at least 80% homology to sequence selected from the group consisting of SEQ ID NOs: 52-56.

21. (canceled)

22. The method of claim 15, wherein the pharmaceutical composition inhibits beta-catenin (β-catenin) association with Transducin Beta-like protein 1 (TBL1) to disrupt formation of a β-catenin:TBL1 complex in the subject; and/or
wherein the pharmaceutical composition comprises one or more pharmaceutically acceptable excipients; and/or
wherein administering the pharmaceutical composition to the subject is selected from the group consisting of intranasal administration, inhalational administration, intravenous administration, oral administration, and parenteral administration.

23-24. (canceled)

25. The method of claim 15, wherein the subject is human.

26. The method of claim 15, wherein the disease is a Wnt/β-catenin active cancer.

27. The method of claim 26, further comprising determining that the subject has a Wnt/β-catenin active cancer; and/or
wherein the Wnt/β-catenin active cancer is selected from the group consisting of colon cancer, colorectal cancer, squamous cell carcinoma, gastric cancer, renal cancer, breast cancer, lung cancer, leukemia, prostate cancer, skin cancer, liver cancer, breast cancer, ovarian cancer, brain cancer and parathyroid cancer; and the first peptide is in an amount sufficient to disrupt the interaction between TBL1 and β-catenin.

28. (canceled)

29. A pharmaceutical composition for the treatment of cancer, the pharmaceutical composition comprising:
a first peptide, wherein the first peptide comprises a first amino acid sequence selected from the group consisting of SEQ ID NOs: 1-33, 52-65, and function-conservative variants thereof, optionally,
further comprising at least one pharmaceutically acceptable carrier.

30. The pharmaceutical composition of claim 29, further comprising a second peptide, wherein the second peptide comprises a second amino acid sequence selected from the group consisting of SEQ ID NOs: 1-33, 52-65, and function-conservative variants thereof;
with the proviso that the first peptide and the second peptide do not comprise the same amino acid sequence, optionally,
further comprising at least one pharmaceutically acceptable carrier.

31. The pharmaceutical composition of claim 30, further comprising a third peptide, wherein the third peptide comprises a third amino acid sequence selected from the group consisting of SEQ ID NOs: 1-33, 52-65, and function-conservative variants thereof;
with the proviso that the first peptide, the second peptide, and the third peptide do not comprise the same amino acid sequence, optionally,
further comprising at least one pharmaceutically acceptable carrier.

32. (canceled)


SEQUENCE LISTING (65 sequences)

SEQ ID NO: 1 - protein, 20 aa, Synthetic construct
INYQDDAELATRAIPELTKL

SEQ ID NO: 2 - protein, 20 aa, Synthetic construct
AIPELTKLLNDEDQVVVNKA

SEQ ID NO: 3 - protein, 20 aa, Synthetic construct
DQVVVNKAAVMVHQLSKKEA

SEQ ID NO: 4 - protein, 20 aa, Synthetic construct
HQLSKKEASRHAIMRSPQMV

SEQ ID NO: 5 - protein, 20 aa, Synthetic construct
IMRSPQMVSAIVRTMQNTND

SEQ ID NO: 6 - protein, 20 aa, Synthetic construct
RTMQNTNDVETARCTAGTLH

SEQ ID NO: 7 - protein, 20 aa, Synthetic construct
RCTAGTLHNLSHHREGLLAI

SEQ ID NO: 8 - protein, 20 aa, Synthetic construct
HREGLLAIFKSGGIPALVKM

SEQ ID NO: 9 - protein, 20 aa, Synthetic construct
GIPALVKMLGSPVDSVLFYA

SEQ ID NO: 10 - protein, 20 aa, Synthetic construct
VDSVLFYAITTLHNLLLHQE

SEQ ID NO: 11 - protein, 20 aa, Synthetic construct
HNLLLHQEGAKMAVRLAGGL

SEQ ID NO: 12 - protein, 20 aa, Synthetic construct
AVRLAGGLQKMVALLNKTNV

SEQ ID NO: 13 - protein, 20 aa, Synthetic construct
ALLNKTNVKFLAITTDCLQI

SEQ ID NO: 14 - protein, 20 aa, Synthetic construct
ITTDCLQILAYGNQESKLII

SEQ ID NO: 15 - protein, 20 aa, Synthetic construct
NQESKLIILASGGPQALVNI

SEQ ID NO: 16 - protein, 20 aa, Synthetic construct
GPQALVNIMRTYTYEKLLWT

SEQ ID NO: 17 - protein, 20 aa, Synthetic construct
TYEKLLWTTSRVLKVLSVCS

SEQ ID NO: 18 - protein, 20 aa, Synthetic construct
LKVLSVCSSNKPAIVEAGGM

SEQ ID NO: 19 - protein, 20 aa, Synthetic construct
AIVEAGGMQALGLHLTDPSQ

SEQ ID NO: 20 - protein, 20 aa, Synthetic construct
LHLTDPSQRLVQNCLWTLRN

SEQ ID NO: 21 - protein, 20 aa, Synthetic construct
NCLWTLRNLSDAATKQEGME

SEQ ID NO: 22 - protein, 20 aa, Synthetic construct
ATKQEGMEGLLGTLVQLLGS

SEQ ID NO: 23 - protein, 20 aa, Synthetic construct
TLVQLLGSDDINVVTCAAGI

SEQ ID NO: 24 - protein, 20 aa, Synthetic construct
VVTCAAGILSNLTCNNYKNK

SEQ ID NO: 25 - protein, 20 aa, Synthetic construct
TCNNYKNKMMVCQVGGIEAL

SEQ ID NO: 26 - protein, 20 aa, Synthetic construct
QVGGIEALVRTVLRAGDRED

SEQ ID NO: 27 - protein, 13 aa, Synthetic construct
LRAGDREDITEPA

SEQ ID NO: 28 - protein, 20 aa, Synthetic construct
LRAGDREDITEPAICALRHL

SEQ ID NO: 29 - protein, 20 aa, Synthetic construct
AICALRHLTSRHQEAEMAQN

SEQ ID NO: 30 - protein, 20 aa, Synthetic construct
QEAEMAQNAVRLHYGLPVVV

SEQ ID NO: 31 - protein, 20 aa, Synthetic construct
HYGLPVVVKLLHPPSHWPLI

SEQ ID NO: 32 - protein, 21 aa, Synthetic construct
PPSHWPLIKATVGLIRNLALC

SEQ ID NO: 33 - protein, 20 aa, Synthetic construct
LIRNLALCPANHAPLREQGA

SEQ ID NO: 34 - protein, 20 aa, Synthetic construct
DVDTSQVLYEWEQGFSQSFT

SEQ ID NO: 35 - protein, 20 aa, Synthetic construct
QGFSQSFTQEQVADIDGQYA

SEQ ID NO: 36 - protein, 23 aa, Synthetic construct
ADIDGQYAMTRAQRVRAAMFPET

SEQ ID NO: 37 - protein, 781 aa, Synthetic construct
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 38 - protein, 393 aa, Synthetic construct
INYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGA

SEQ ID NO: 39 - protein, 19 aa, Synthetic construct
VDSVLFAITTLHNLLLHQE

SEQ ID NO: 40 - protein, 20 aa, Synthetic construct
HNLLLHQEGAKMAVRLAGGL

SEQ ID NO: 41 - protein, 20 aa, Synthetic construct
ALLNKTNVKFLAITTDCLQI

SEQ ID NO: 42 - protein, 20 aa, Synthetic construct
ITTDCLQILAYGNQESKLII

SEQ ID NO: 43 - protein, 20 aa, Synthetic construct
NQESKLIILASGGPQALVNI

SEQ ID NO: 44 - protein, 20 aa, Synthetic construct
GPQALVNIMRTYTYEKLLWT

SEQ ID NO: 45 - protein, 20 aa, Synthetic construct
TYEKLLWTTSRVLKVLSVCS

SEQ ID NO: 46 - protein, 20 aa, Synthetic construct
LKVLSVCSSNKPAIVEAGGM

SEQ ID NO: 47 - protein, 20 aa, Synthetic construct
LHLTDPSQRLVQNCLWTLRN

SEQ ID NO: 48 - protein, 20 aa, Synthetic construct
NCLWTLRNLSDAATKQEGME

SEQ ID NO: 49 - protein, 20 aa, Synthetic construct
ATKQEGMEGLLGTLVQLLGS

SEQ ID NO: 50 - protein, 20 aa, Synthetic construct
TLVQLLGSDDINVVTCAAGI

SEQ ID NO: 51 - protein, 20 aa, Synthetic construct
VVTCAAGILSNLTCNNYKNK

SEQ ID NO: 52 - protein, 44 aa, Synthetic construct
HQLSKKEASRHAIMRSPQMVSAIVRTMQNTNDVETARCTAGTLH

SEQ ID NO: 53 - protein, 44 aa, Synthetic construct
VDSVLFYAITTLHNLLLHQEGAKMAVRLAGGLQKMVALLNKTNV

SEQ ID NO: 54 - protein, 44 aa, Synthetic construct
NQESKLIILASGGPQALVNIMRTYTYEKLLWTTSRVLKVLSVCS

SEQ ID NO: 55 - protein, 44 aa, Synthetic construct
LKVLSVCSSNKPAIVEAGGMQALGLHLTDPSQRLVQNCLWTLRN

SEQ ID NO: 56 - protein, 44 aa, Synthetic construct
VVTCAAGILSNLTCNNYKNKMMVCQVGGIEALVRTVLRAGDRED

SEQ ID NO: 57 - protein, 25 aa, Synthetic construct
CGRKKRRQRRRPPQCNQESKLIILA

SEQ ID NO: 58 - protein, 25 aa, Synthetic construct
CGRKKRRQRRRPPQCLIILASGGPQ

SEQ ID NO: 59 - protein, 25 aa, Synthetic construct
CGRKKRRQRRRPPQCSGGPQALVNI

SEQ ID NO: 60 - protein, 25 aa, Synthetic construct
CGRKKRRQRRRPPQCALVNIMRTYT

SEQ ID NO: 61 - protein, 25 aa, Synthetic construct
CGRKKRRQRRRPPQCMRTYTYEKLL

SEQ ID NO: 62 - protein, 25 aa, Synthetic construct
CGRKKRRQRRRPPQCYEKLLWTTSR

SEQ ID NO: 63 - protein, 25 aa, Synthetic construct
CGRKKRRQRRRPPQCWTTSRVLKVL

SEQ ID NO: 64 - protein, 25 aa, Synthetic construct
CGRKKRRQRRRPPQCVLKVLSVASS

SEQ ID NO: 65 - protein, 25 aa, Synthetic construct
CGRKKRRQRRRPPQCAEISLIQKLN